(19) United States
(12) Patent Application Publication
(10) Pub. No.: US 2026/0217554 A1
(43) Pub. Date: Jul. 30, 2026

(54) Surface Coatings for Enhanced Substrate Properties

(71) Applicant: Temple University - Of The Commonwealth System of Higher Education, Philadelphia, PA (US)

(72) Inventors: Rouzbeh Afsarmanesh Tehrani, Emeryville, CA (US); Farbod Alimohammadi, Philadelphia, PA (US)

(21) Appl. No.: 19/146,973

(22) PCT Filed: Jan. 10, 2024
(86) PCT No.: PCT/US2024/011049

§ 371 Date: Jul. 10, 2025

Related U.S. Application Data
(60) Provisional application No. 63/479,288, filed on Jan. 10, 2023.

(51) Int. Cl. C01G 45/024; A61L 2/232; A61L 31/08; A61L 31/10; A61L 31/14; A61L 31/16; A61L 101/02; A61L 103/15; A61M 1/16; B01J 19/02; B01J 19/24; B01J 20/06; B01J 20/10; B01J 20/20; B01J 20/32; C02F 1/28; C02F 101/20; C09D 5/14; C09D 5/16; C09D 7/61; C09D 171/02

(52) U.S. Cl. CPC C01G 45/024; A61L 2/232; A61L 31/088; A61L 31/10; A61L 31/146; A61L 31/16; A61M 1/1627; B01J 19/02; B01J 19/24; B01J 20/06; B01J 20/103; B01J 20/20; B01J 20/3204; B01J 20/321; B01J 20/3214; B01J 20/3236; C02F 1/281; C09D 5/14; C09D 5/1618; C09D 7/61; C09D 171/02; A61L 2101/02; A61L 2103/15; A61L 2300/406; A61L 2420/02; A61L 2420/08; B01J 2219/0218; C01P 2002/85; C01P 2004/03; C02F 2101/20

(57) ABSTRACT

The present invention relates to the discovery that a uniform coating of MnO₂ can prevent bacterial contamination. Accordingly, the invention includes methods of producing one or more MnO₂ coating on a surface. The invention also includes methods of preventing bacterial contamination on a surface by applying the specified coating.

### CROSS-REFERENCE TO RELATED APPLICATIONS

The present application claims priority to U.S. Provisional Application No. 63/479,288, filed on Jan. 10, 2023, the contents of which are hereby incorporated by reference in their entirety.

### BACKGROUND OF THE INVENTION

Given the global SARS-CoV2 pandemic, public hygiene has undergone an increase in scrutiny. One aspect of public hygiene that has attracted particular attention are high-touch surfaces, such as doors, faucets, counters, publicly accessible electronics, and the like. In response to this, surface coatings capable of minimizing pathogen reproduction and stability have been explored for their applications in any number of settings. Among the surface coatings being examined is manganese dioxide (MnO₂).

Although MnO₂ surface coatings have demonstrated promising results as antibacterial coatings, and removal of heavy metals from the environment, there has been minimal improvement in methods for producing MnO₂-coated materials. Common methods for coating materials in MnO₂ require synthesis of a coating material followed by an application of the coating material, necessitating multiple steps for preparation. Other methods involving dispersion of MnO₂ into polymers or onto surfaces make it difficult to achieve a uniform coating or control the thickness of the coating. These methods are also often only amenable to a select number of materials with easily accessible surfaces.

Therefore, there is a need for the development of a single-step, easily controlled method of applying a MnO₂ coating to a variety of substrates, even with complex surfaces. The present invention addresses this long needed but unmet requirement.

### SUMMARY OF THE INVENTION

In some embodiments, the present invention provides a method of forming an essentially uniform coating of manganese (IV) oxide (MnO₂) on a surface comprising: a) contacting the surface in a solution of one or more manganese (II) salts; b) contacting the surface in a solution of one or more permanganate salts; and c) repeating steps a) and b) until the MnO₂ coating is of the desired thickness. In some embodiments, the solution of one or more manganese (II) salts is a solution of manganese (II) nitrate (Mn(NO₃)₂). In some embodiments, the solution of Mn(NO₃)₂ is at a concentration of about 20 g/L. In some embodiments, the solution of one or more permanganate salts is a solution of potassium permanganate (KMnO₄). In some embodiments, the solution of KMnO₄ is at a concentration of about 20 g/L.

In some embodiments, the solutions of steps a) and b) are at about 50° C. In some embodiments, the solutions of steps a) and b) are aqueous solutions. In some embodiments, the solution of one or more manganese (II) salts has one or more Mn²⁺ concentration of about 0.11 M. In some embodiments, the solution of permanganate solution has one or more MnO₄ concentration of about 0.13 M.

In some embodiments, the surface comprises one or more selected from the group consisting of plastic, glass, and metal. In some embodiments, the plastic is polyvinyl chloride (PVC). In some embodiments, the metal is copper.

In some embodiments, the disclosure provides a method of forming an essentially uniform coating of manganese (IV) oxide (MnO₂) on a surface comprising: a) contacting the surface with a solution comprising one or more manganese (II) salts, one or more permanganate salts, and one or more stabilizers; b) allowing the surface to dry; and c) repeating steps a) and b) until the coating reaches the desired thickness. In some embodiments, the one or more Mn (II) salt is Mn(NO₃)₂. In some embodiments, the one or more permanganate salt is KMnO₄. In some embodiments, the one or more stabilizers are one or more surfactants and/or polymers. In some embodiments, wherein the one or more polymers is a polyethylene glycol (PEG)-polypropylene glycol (PPG)-PEG triblock copolymer.

In some embodiments, step a) further comprises exposing the solution to microwaves until the solution reaches a temperature of about 60° C. before contacting the surface with the solution. In some embodiments, the method further comprises maintaining the solution at about 60° C. for about 30 minutes before contacting the surface with the solution. In some embodiments, the method further comprises cooling the solution from 60° C. to about room temperature before contacting the surface with the solution.

In some embodiments, the method further comprises applying to the surface one or more carbon-based adsorbents, wherein the carbon-based adsorbent is one or more selected from the group consisting of carbon, charcoal, graphene, graphene oxide, graphitic composites, carbon tubes, and carbon fibers. In some embodiments, the method further comprises applying to the surface one or more biocompatible polymers or resins, wherein the polymer or resin is selected form the group consisting of cyanoacrylates, fibrinogen, thrombin), polyethylene glycol (PEG), silicone, polyurethanes, methyl methacrylate, gelatin-resorcinol-formaldehyde (GRF) glue, albumin, glutaraldehyde, acrylics, and epoxy resins.

In some embodiments, the present disclosure provides a method of producing an antibacterial coating on a surface comprising treating a surface according to a method of the present invention.

In some embodiments, the present disclosure provides a method of preventing or minimizing bacterial contamination on a surface comprising covering the surface with an antibacterial coating according to a method of the present invention.

In some embodiments, the present disclosure provides a method of reducing heavy metal contamination in an aqueous environment comprising placing an object with surfaces treated according to the present invention in the aqueous environment. In some embodiments, the heavy metal is lead.

In some embodiments, the present disclosure provides a plug flow reactor (PFR), wherein the interior surfaces of the pipes, tubes, and/or lines of the PFR are partially or fully coated with an MnO₂ coating produced according to a method of the present invention. In some embodiments, the PFR further comprises a static mixer. In some embodiments, the surface of the static mixer is partially or fully coated with an MnO₂ coating produced according to a method of the present invention.

In some embodiments, the present invention provides a PFR, wherein the PFR comprises a static mixer, wherein the surface of the static mixer is partially or fully coated with an MnO₂ coating produced according to a method of the present invention.

In some embodiments, the present invention provides a dialysis line, wherein the inner surface of the dialysis tube is coated with an adsorbent layer and the inner surface of the adsorbent layer is coated with a biocompatible porous layer. In some embodiments, the adsorbent layer comprises one or more of: a) an MnO₂ coating produced according to the method of any one of claims 1, 13, 21, and 22; b) one or more selected from the group consisting of activated charcoal, amberlite, XAD resins, zeolites, silica gel, polyacrylate hydrogels, cellulose acetate-coated activated charcoal, chitosan, activated alumina, modified graphite, and graphene; and c) an adsorptive polymer. In some embodiments, the adsorptive polymer is selected from the group consisting of sevelamer, polystyrene sulfonate, and cholestyramine. In some embodiments, the biocompatible porous layer comprises one or more selected from the group consisting of: polystyrene, polypropylene, polyvinylchloride, polyethylene, polyurethane, polycarbonate, polyethylene terephthalate, polyether ether ketone, PEG, PPG, polysulfone (PSU), polyethersulfone (PES), polyacrylonitrile (PAN), polyamide, cellulose acetate, modified cellulose, hemophan, polyvinylpyrrolidone (PVP), and poly(methyl methacrylate) (PMMA), polyethylene glycol diacrylate, poly(lactic-co-glycolic acid) (PLGA), polydimethylsiloxane (PDMS), polyvinyl alcohol (PVA), polyacrylamide, polytetrafluoroethylene, and combinations thereof. In some embodiments, the polymer further comprises heparain. In some embodiments, the biocompatible porous layer has an average pore size between about 10 nm and about 6 μm. In some embodiments, the adsorbent layer is impregnated with one or more active agents. In some embodiments, the one or more active agents are one or more selected from the group consisting of: vitamins, minerals, ions, amino acids, analgesics, anesthetics, antibiotics, antivirals, anti-helminthics, anti-fungals, anticoagulants, anti-cancer agents, proteins, and antibodies. In some embodiments, the one or more antibodies are selected from the group consisting of basiliximab, daclizumab, infliximab, and intravenous immunoglobulin (IVIG).

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 depicts a representative image of a number of representative materials coated with δ-MnO₂, as well as SEM images of MnO₂ coatings on two different glass substrates.

FIG. 2 depicts representative images of static mixers coated with δ-MnO₂ using a microwave synthesis method from above (top) and the side (bottom) showing the nano-knife effect.

FIG. 3, comprising FIG. 3A through FIG. 3D, depicts representative images of static mixers coated with δ-MnO₂ and activated carbon. FIG. 3A depicts a representative image of static mixers coated with δ-MnO₂ or δ-MnO₂ and activated carbon prepared using a microwave synthesis. FIG. 3B depicts representative SEM images of a static mixer coated with δ-MnO₂ and activated carbon using a microwave synthesis. FIG. 3C depicts representative images of a static mixer coated with δ-MnO₂ and activated carbon using a microwave synthesis. FIG. 4D depicts representative images of a static mixer coated with δ-MnO₂ and activated carbon using a dip method.

FIG. 4, comprising FIG. 4A through FIG. 4D, depicts representative images of a high-density polyethylene (HDPE) mixer coated with MnO₂ for adsorption of lead. FIG. 4A depicts a representative image of an HDPE mixer which has partially been coated with MnO₂. FIG. 4B depicts a representative scanning electron microscopy image of the coated HDPE mixer depicted in FIG. 4A at the interface of the coated and uncoated areas. Scale bar=5 mm. FIG. 4C depicts a representative scanning electron microscopy image of the coated HDPE mixer after being used to treat lead-containing water. Scale bar=1 mm. FIG. 4D depicts representative energy-dispersive X-ray spectroscopy (EDS) analysis results for a section of the coated HDPE mixer after being used to treat lead-containing water, as seen in FIG. 4C.

FIG. 5, comprising FIG. 5A through FIG. 5C, depicts representative results of elemental analysis of MnO₂-silica composite coating before incubation in lead-contaminated water. FIG. 5A depicts a representative SEM image of an HDPE mixer coated with MnO₂-silica composite before incubation in lead-contaminated water. Two fields of analysis for EDS analysis. FIG. 5B depicts EDS analysis results for Spectrum 7 as outlined in FIG. 5A. FIG. 5C depicts EDS analysis results for Spectrum 9 as outlined in FIG. 5A.

FIG. 6, comprising FIG. 6A through FIG. 6C, depicts representative results of elemental analysis of MnO₂-silica composite coating before incubation in lead-contaminated water. FIG. 6A depicts a representative SEM image of an HDPE mixer coated with MnO₂-silica composite before incubation in lead-contaminated water. Two fields of analysis for EDS analysis. FIG. 6B depicts EDS analysis results for Spectrum 4 as outlined in FIG. 6A. FIG. 6C depicts EDS analysis results for Spectrum 5 as outlined in FIG. 6A.

FIG. 7 depicts a representative image of a rotary reactor setup with a MnO₂-coated HDPE mixer used for measuring the antibacterial properties of the MnO₂ coating.

FIG. 8, comprising FIG. 8A through FIG. 8D, depicts representative images of hydrophobicity analysis of MnO₂ coatings. FIG. 8A depicts representative images of contact angle analysis various surface coatings, from superhydrophobic (top) to hydrophilic (bottom). FIG. 8B depicts representative images of PVC pipe coated with an MnO₂ polymeric composite by dip coating, with a scratch to reveal the underlying PVC surface (left) and after the interior has been exposed to lead-contaminated water (right). FIG. 8C depicts a representative SEM image of MnO₂ polymeric composite scraped off of a coated PVC pipe. FIG. 8D depicts a representative image of an MnO₂ polymeric composite showing the adhesive side that is adjacent to the PVC pipe.

FIG. 9 depicts representative raw data for contact angle measurement of water on various MnO₂-adhesive coatings. Summarized values are in Table 2.

FIG. 10, comprising FIG. 10A through FIG. 10C, depicts representative images of computational flow dynamics (CFD) modeling of a plug flow reactor with a length of 100 mm and an inner diameter of 8 mm when exposed to 4 mol/m³ contaminant. FIG. 10A depicts a representative image of CFD modeling for concentration of contaminant as it is adsorbed by the MnO₂ coating, with the concentration of contaminant dropping from 4 mol/m³ to 2-2.8 mol/m³. FIG. 10B depicts a representative image of CFD modeling for laminar flow rate in an MnO₂ coated PFR with fluid entering the PFR with an initial linear velocity of 0.09 m/s. FIG. 10C depicts a schematic of a PFR coated with an adsorbent and a representative decrease in contaminant concentration over the length of the coated PFR.

FIG. 11, comprising FIG. 11A through FIG. 11E, depicts representative images of CFD modeling for a PFR, as in FIG. 10, with a static mixer. FIG. 11A depicts a representative image of a coated helical static mixer installed in a pipe. FIG. 11B depicts a representative image of CFD modeling for a fluid, with an initial concentration of 5 mol/m³, flowing into the adsorptive static mixer from bottom to top. FIG. 11C depicts a representative image of a cross-sectional view of FIG. 11B, with adsorptive reactions occurring on the coated interior surfaces, while the static mixer facilitates mixing of the fluid. FIG. 11D depicts a representative image of CFD modeling demonstrating fluid entry into an adsorptive static mixer with an initial concentration of 5 mol/m³ flowing left to right.

FIG. 12 depicts a schematic representation of a cross-section of a surface coated dialysis tubing, with a porous layer of a biocompatible material separating the adsorbent from directly contacting cells in the bodily fluid.

FIG. 13 depicts representative modeling of toxin concentration resulting from blood flowing through a dialysis line shown in FIG. 12 with toxins being adsorbed onto the adsorbent coating as the blood flows through the tube.

FIG. 14 depicts representative images of biogenically synthesized magnetic and paramagnetic iron oxide and lanthanide composites.

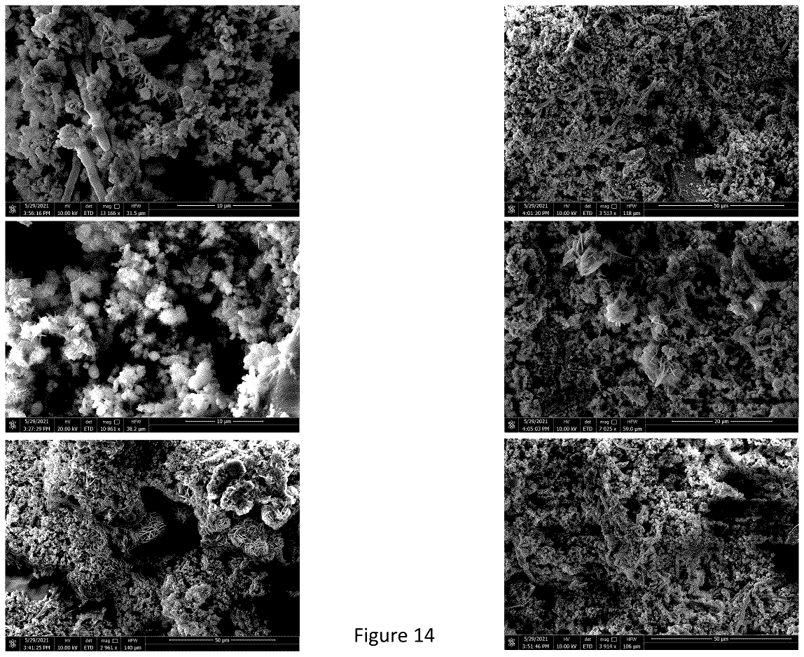

### DETAILED DESCRIPTION

The present invention is based partly on the discovery that a uniform coating of MnO₂ on a surface possesses antibacterial and heavy metal adsorption properties. Therefore, in various embodiments, the invention relates to methods imbuing of antibacterial properties to surfaces by coating them with MnO₂. In other embodiments, the invention relates to making surfaces capable of adsorbing heavy metals by coating them with MnO₂. Accordingly, in other embodiments, the invention relates to methods of using surfaces coated with MnO₂ for removal and sequestration of heavy metals from the environment.

In another aspect, the invention is partly based on a discovery that sequential contacting of a surface in solutions of one or more Mn²⁺ salts and one or more MnO₄ salts can produce a uniform coating of MnO₂ of a controllable thickness. Therefore, in some embodiments, the invention relates to methods of producing one or more MnO₂ coating on a surface by contacting it solutions of one or more Mn²⁺ salts and one or more MnO₄ salts. In some embodiments, the method comprises contacting the surface in a solution of Mn(NO₃)₂. In some embodiments, the method comprises contacting the surface in a solution of KMnO₄. In some embodiments, the method comprises contacting the surface in manganese solutions at 50° C.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

“Hydrophobic” surfaces, as described herein, are characterized by having a contact angle, formed by the surface and a drop or droplet of water, between 90° and 180°. A “superhydrophobic” surface is a hydrophobic surface with a contact angle greater than about 120°, greater than about 130°, greater than about 140°, or greater than about 150°. Surfaces that are “hydrophilic” have a contact angle less than 90° or may not have a measurable contact angle as the drop or droplet of water is absorbed into the surface. The volume of the drop or droplet of water for measuring a contact angle should generally be between about 5 μL and about 20 μL, preferably about 10 μL, although an exact volume is not required for measurement.

“Measuring” or “measurement,” or alternatively “detecting” or “detection,” means assessing the presence, absence, quantity or amount (which can be an effective amount) of either a given substance within a sample, including the derivation of qualitative or quantitative concentration levels of such substances, or otherwise evaluating the values or categorization of the substance or the sample.

A “plug flow reactor” or “PFR” is a type of chemical reactor that embodies a tubular design, through which a fluid (either gas or liquid) flows continuously at a steady rate. Characterized by its ability to maintain a spatially uniform velocity profile, the PFR operates under the premise that fluid elements retain their identity without back-mixing, essentially moving as discrete “plugs” through the reactor. This feature results in a predictable and uniform residence time for each reactant element, facilitating the control of reaction times and efficiencies. Ideal PFRs exhibit a linear concentration gradient along the reactor's length, with reaction kinetics typically following first-order dynamics, making them particularly suitable for reactions with a constant rate per unit of reactor volume.

A “static mixer” is a precision-engineered device for continuously mixing fluid materials, without moving parts. By continuously blending fluids within a pipeline, the static mixer utilizes a series of fixed elements that repeatedly divide, reorient, and combine the flow materials to achieve homogeneity. As fluid flows through the mixer, the non-moving elements create specific flow patterns that ensure thorough mixing. This design is highly efficient for mixing liquids, gases, or a combination, and is particularly advantageous in processes where low energy consumption, consistent product quality, and minimal maintenance are crucial. Static mixers are widely used in various industries, including chemical processing, food and beverage, and water treatment, due to their compact size, ease of installation, and ability to handle various viscosities and flow conditions.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the discovery that coating a surface with uniform layers of MnO₂ imbues the surface with antibacterial and heavy metal-adsorbing properties. Accordingly, in various embodiments the invention is directed towards methods of coating a surface with a uniform layer of MnO₂.

In certain embodiments, the invention is directed towards antibacterial surfaces coated with MnO₂ and methods of making antibacterial surfaces.

In other embodiments, the invention is directed towards making surfaces capable of adsorbing heavy metals from the environment. In other embodiments, the invention is directed towards methods of using a surface coated in MnO₂ to remove heavy metals from the environment.

### Methods of Coating a Surface

The present invention provides methods for coating a surface with a uniform layer of MnO₂.

In various embodiments, the method comprises obtaining a surface to be coated with MnO₂. In one embodiment, the method comprises contacting the surface with a solution of one or more Mn²⁺ salts and contacting the surface with a solution of one or more MnO₄ salts. In various embodiments, the method comprises repeatedly contacting the surface the solutions in order to form a layer of MnO₂ of a desired thickness.

In some embodiments, the method comprises repeating the steps of contacting the surface with a solution of one or more Mn²⁺ salts and a solution of one or more MnO₄ salts between one and 100 times. In some embodiments, the method comprises repeating the steps between five and 50 times. In some embodiments, the method comprises repeating the steps between 10 and 25 times. In some embodiments, the method comprises repeating the steps between 15 and 20 times. In some embodiments, the method comprises repeating the step one, five, 10, 15, 20, 25, 50, or 100 times.

In some embodiments, the method comprises repeating the steps of contacting the surface with a solution of one or more Mn²⁺ salts and a solution of one or more MnO₄ salts until the layer of MnO₂ reaches a desired thickness. In some embodiments, the steps are repeated until the layer of MnO₂ is between about 0.1 nm and about 1 mm. In some embodiments, the steps are repeated until the layer of MnO₂ is between about 1 nm and about 100 μm. In some embodiments, the steps are repeated until the layer of MnO₂ is between about 10 nm and about 10 μm. In some embodiments, the steps are repeated until the layer of MnO₂ is between about 100 nm and about 1000 nm. In some embodiments, the steps are repeated until the layer of MnO₂ is between about 200 nm and about 500 nm. In some embodiments, the steps are repeated until the layer of MnO₂ is between about 300 nm and about 400 nm. In some embodiments, the steps are repeated until the layer of MnO₂ is about 0.1 nm, about 1 nm, about 10 nm, about 100 nm, about 1 μm, about 10 μm, about 100 μm, or about 1 mm in thickness.

In some embodiments, the method comprises the step of rinsing the surface with water. In some embodiments, the water is tap water, filtered water, distilled water, or deionized water.

In some embodiments, the surface is contacted in a solution of one or more Mn²⁺ salts for between about 0.1 s and about 1 h. In some embodiments, the surface is contacted for between about 1 s and about 30 min. In some embodiments, the surface is contacted for between about 10 s and about 3 min. In some embodiments, the surface is contacted for between about 30 s and about 60 s. In some embodiments, the surface is contacted for between about 40 s and about 50 s. In some embodiments, the surface is contacted for about 0.1 s, about 1 s, about 10 s, about 30 s, about 40 s, about 45 s, about 50 s, about 60 s, about 3 min, about 30 min, or about 1 h.

In some embodiments, the solution of one or more Mn²⁺ salts has a concentration of Mn²⁺ between about 0.01 M and about 6.5 M. In some embodiments, the concentration is between about 0.05 M and about 1 M. In some embodiments, the concentration is between about 0.05 M and about 0.5 M. In some embodiments, the concentration is between about 0.075 M and about 0.5 M. In some embodiments, the concentration is between about 0.1 M and about 0.25 M. In some embodiments, the concentration is between about 0.1 M and about 0.2 M. In some embodiments, the concentration is between about 0.1 M and about 0.15 M. In some embodiments, the concentration is about 0.05 M, about 0.075 M, about 0.1 M, about 0.11 M, about 0.15 M, about 0.2 M, about 0.25 M, about 0.5 M, about 1 M, about 5 M, or about 6.5 M.

In some embodiments, the solution of one or more Mn²⁺ salts is a solution of one or more selected from the group consisting of manganese (II) nitrate (Mn(NO₃)₂), manganese (II) sulfate (MnSO₄), manganese (II) chloride (MnCl₂), manganese (II) bromide (MnBr₂), manganese (II) iodide (MnI₂), and manganese (II) acetate (Mn(CH₃CO₂)₂. In some embodiments, the solution is a solution of Mn(NO₃)₂.

In some embodiments, the solution of Mn(NO₃)₂ is at a concentration of between about 1.8 g/L and about 1,160 g/L. In some embodiments, the concentration is between about 9 g/L and about 180 g/L. In some embodiments, the concentration is between about 13.5 g/L and about 90 g/L. In some embodiments, the concentration is between about 18 g/L and about 45 g/L. In some embodiments, the concentration is between about 18 g/L and about 36 g/L. In some embodiments, the concentration is between about 18 g/L and about 27 g/L. In some embodiments, the concentration is about 9 g/L, about 13.5 g/L, about 18 g/L, about 20 g/L, about 27 g/L, about 45 g/L, about 45 g/L, about 90 g/L, about 180 M, about 895 g/L, or about 1,160 g/L.

In some embodiments, the surface is contacted in a solution of one or more MnO₄⁻ salts for between about 0.1 s and about 1 h. In some embodiments, the surface is contacted for between about 1 s and about 30 min. In some embodiments, the surface is contacted for between about 10 s and about 3 min. In some embodiments, the surface is contacted for between about 30 s and about 60 s. In some embodiments, the surface is contacted for between about 40 s and about 50 s. In some embodiments, the surface is contacted for about 0.1 s, about 1 s, about 10 s, about 30 s, about 40 s, about 45 s, about 50 s, about 60 s, about 3 min, about 30 min, or about 1 h.

In some embodiments, the solution of one or more MnO₄⁻ salts has at a concentration of MnO₄⁻ between about 0.01 M and about 10 M. In some embodiments, the concentration is between about 0.05 M and about 1 M. In some embodiments, the concentration is between about 0.05 M and about 0.5 M. In some embodiments, the concentration is between about 0.075 M and about 0.5 M. In some embodiments, the concentration is between about 0.1 M and about 0.25 M. In some embodiments, the concentration is between about 0.1 M and about 0.2 M. In some embodiments, the concentration is between about 0.1 M and about 0.15 M. In some embodiments, the concentration is about 0.05 M, about 0.075 M, about 0.1 M, about 0.13 M, about 0.15 M, about 0.2 M, about 0.25 M, about 0.5 M, about 1 M, about 5 M, or about 10 M.

In some embodiments, the solution of one or more MnO₄⁻ salts is a solution of one or more selected from the group consisting of potassium permanganate (KMnO₄), sodium permanganate (NaMnO₄), ammonium permanganate (NH₄MnO₄), calcium permanganate (Ca(MnO₄)₂), and barium permanganate (Ba(MnO₄)₂). In some embodiments, the solution is a solution of KMnO₄.

In some embodiments, the solution of one or more MnO₄ salts is at a concentration of between about 1.6 g/L and about 1,600 g/L. In some embodiments, the concentration is between about 8 g/L and about 160 g/L. In some embodiments, the concentration is between about 12 g/L and about 80 g/L. In some embodiments, the concentration is between about 16 g/L and about 40 g/L. In some embodiments, the concentration is between about 16 g/L and about 32 g/L. In some embodiments, the concentration is between about 16 g/L and about 24 g/L. In some embodiments, the concentration is about 8 g/L, about 12 g/L, about 16 g/L, about 20 g/L, about 24 g/L, about 32 g/L, about 40 g/L, about 80 g/L, about 160 g/L, about 800 g/L, or about 1,600 g/L.

In some embodiments, the solutions of one or more Mn²⁺ salts and one or more MnO₄⁻ salts are at between about 25° C. and about 100° C. In some embodiments, the solutions are at between about 30° C. and about 80° C. In some embodiments, the solutions are at between about 40° C. and about 60° C. In some embodiments, the solutions are at about 25° C., about 30° C., about 40° C., about 50° C., about 60° C., about 80° C., or about 100° C. In some embodiments, the solutions are at different temperatures.

In some embodiments, the solutions of one or more Mn²⁺ salts and one or more MnO₄⁻ salts are aqueous. In some embodiments, the solutions are in a combination of water and one or more organic solvents. Examples of suitable organic solvents include, but are not limited to, acetone, ethyl acetate, dimethyl sulfoxide, acetonitrile, and dimethylformamide. In some embodiments, the solutions are in different solvents.

In some embodiments, the method comprises obtaining a surface to be coated with MnO₂. In one embodiment, the method comprises contacting the surface with a solution comprising one or more Mn²⁺ salts, one or more MnO₄⁻ salts, and one or more stabilizers. In various embodiments, the method comprises repeatedly contacting the surface with the solution in order to form a layer of MnO₂ of a desired thickness.

In some embodiments, the method comprises repeating the steps of contacting the surface with a solution comprising one or more Mn²⁺ salts, one or more MnO₄⁻ salts, and one or more polymers between one and 100 times. In some embodiments, the method comprises repeating the steps between five and 50 times. In some embodiments, the method comprises repeating the steps between 10 and 25 times. In some embodiments, the method comprises repeating the steps between 15 and 20 times. In some embodiments, the method comprises repeating the steps one, five, 10, 15, 20, 25, 50, or 100 times.

In some embodiments, the method comprises repeating the step of contacting the surface with a solution comprising one or more Mn²⁺ salts, one or more MnO₄⁻ salts, and one or more polymers until the layer of MnO₂ reaches a desired thickness. In some embodiments, the steps are repeated until the layer of MnO₂ is between about 0.1 nm and about 1 mm. In some embodiments, the steps are repeated until the layer of MnO₂ is between about 1 nm and about 100 μm. In some embodiments, the steps are repeated until the layer of MnO₂ is between about 10 nm and about 10 μm. In some embodiments, the steps are repeated until the layer of MnO₂ is between about 100 nm and about 1000 nm. In some embodiments, the steps are repeated until the layer of MnO₂ is between about 200 nm and about 500 nm. In some embodiments, the steps are repeated until the layer of MnO₂ is between about 300 nm and about 400 nm. In some embodiments, the steps are repeated until the layer of MnO₂ is about 0.1 nm, about 1 nm, about 10 nm, about 100 nm, about 1 μm, about 10 μm, about 100 μm, or about 1 mm in thickness.

In some embodiments, the method comprises the step of rinsing the surface with water. In some embodiments, the water is tap water, filtered water, distilled water, or deionized water.

In some embodiments, the surface is contacted in a solution comprising one or more Mn²⁺ salts, one or more MnO₄⁻ salts, and one or more polymers for between about 0.1 s and about 1 h. In some embodiments, the surface is contacted for between about 1 s and about 30 min. In some embodiments, the surface is contacted for between about 10 s and about 3 min. In some embodiments, the surface is contacted for between about 30 s and about 60 s. In some embodiments, the surface is contacted for between about 40 s and about 50 s. In some embodiments, the surface is contacted for about 0.1 s, about 1 s, about 10 s, about 30 s, about 40 s, about 45 s, about 50 s, about 60 s, about 3 min, about 30 min, or about 1 h.

In some embodiments, the solution comprises one or more Mn²⁺ salts at a concentration of Mn²⁺ between about 0.01 M and about 6.5 M. In some embodiments, the concentration is between about 0.05 M and about 1 M. In some embodiments, the concentration is between about 0.05 M and about 0.5 M. In some embodiments, the concentration is between about 0.075 M and about 0.5 M. In some embodiments, the concentration is between about 0.1 M and about 0.25 M. In some embodiments, the concentration is between about 0.1 M and about 0.2 M. In some embodiments, the concentration is between about 0.1 M and about 0.15 M. In some embodiments, the concentration is about 0.05 M, about 0.075 M, about 0.9 M, about 0.1 M, about 0.11 M, about 0.15 M, about 0.2 M, about 0.25 M, about 0.5 M, about 1 M, about 5 M, or about 6.5 M.

In some embodiments, the one or more Mn²⁺ salts are one or more selected from the group consisting of manganese (II) nitrate (Mn(NO₃)₂), manganese (II) sulfate (MnSO₄), manganese (II) chloride (MnCl₂), manganese (II) bromide (MnBr₂), manganese (II) iodide (MnI₂), and manganese (II) acetate (Mn(CH₃CO₂)₂. In some embodiments, the solution is a solution of Mn(NO₃)₂.

In some embodiments, the solution comprises Mn(NO₃)₂ at a concentration of between about 1.7 g/L and about 1,160 g/L. In some embodiments, the concentration is between about 8.5 g/L and about 170 g/L. In some embodiments, the concentration is between about 13.5 g/L and about 90 g/L. In some embodiments, the concentration is between about 17 g/L and about 45 g/L. In some embodiments, the concentration is between about 17 g/L and about 36 g/L. In some embodiments, the concentration is between about 17 g/L and about 27 g/L. In some embodiments, the concentration is about 9 g/L, about 13.5 g/L, about 14 g/L, about 15 g/L, about 16 g/L, about 17 g/L, about 18 g/L, about 19 g/L, about 20 g/L, about 27 g/L, about 45 g/L, about 45 g/L, about 90 g/L, about 180 g/L, about 895 g/L, or about 1,160 g/L.

In some embodiments, the solution comprises one or more MnO₄⁻ salts at a concentration of MnO₄⁻ between about 0.01 M and about 10 M. In some embodiments, the concentration is between about 0.05 M and about 1 M. In some embodiments, the concentration is between about 0.05 M and about 0.5 M. In some embodiments, the concentration is between about 0.075 M and about 0.5 M. In some embodiments, the concentration is between about 0.1 M and about 0.25 M. In some embodiments, the concentration is between about 0.1 M and about 0.2 M. In some embodiments, the concentration is between about 0.1 M and about 0.15 M. In some embodiments, the concentration is about 0.05 M, about 0.075 M, about 0.08 M, about 0.09 M, about 0.1 M, about 0.11 M, about 0.12 M, about 0.13 M, about 0.15 M, about 0.2 M, about 0.25 M, about 0.5 M, about 1 M, about 5 M, or about 10 M.

In some embodiments, the one or more MnO₄⁻ salts are one or more selected from the group consisting of potassium permanganate (KMnO₄), sodium permanganate (NaMnO₄), ammonium permanganate (NH₄MnO₄), calcium permanganate (Ca(MnO₄)₂), and barium permanganate (Ba(MnO₄)₂). In some embodiments, the solution is a solution of KMnO₄.

In some embodiments, the solution of one or more MnO₄⁻ salts is at a concentration of between about 1.6 g/L and about 1,600 g/L. In some embodiments, the concentration is between about 8 g/L and about 160 g/L. In some embodiments, the concentration is between about 12 g/L and about 80 g/L. In some embodiments, the concentration is between about 16 g/L and about 40 g/L. In some embodiments, the concentration is between about 16 g/L and about 32 g/L. In some embodiments, the concentration is between about 16 g/L and about 24 g/L. In some embodiments, the concentration is about 8 g/L, about 12 g/L, about 16 g/L, about 20 g/L, about 24 g/L, about 32 g/L, about 40 g/L, about 80 g/L, about 160 g/L, about 800 g/L, or about 1,600 g/L.

In some embodiments, the solution comprises one or more stabilizers at a concentration between about 0.6 g/L and about 60 g/L. In some embodiments, the concentration is between about 1.2 g/L and about 30 g/L. In some embodiments, the concentration is between about 2.4 g/L and about 15 g/L. In some embodiments, the concentration is between about 4.8 g/L and about 10 g/L. In some embodiments, the concentration is about 0.6 g/L, about 1.2 g/L, about 1.8 g/L, about 2.4 g/L, about 3 g/L, about 3.6 g/L, about 4.2 g/L, about 4.8 g/L, about 5.4 g/L, about 5.5 g/L, about 5.6 g/L, about 5.7 g/L, about 5.8 g/L, about 5.9 g/L, about 6 g/L, about 6.1 g/L, about 6.2 g/L, about 6.3 g/L, about 6.4 g/L, about 6.5 g/L, about 6.6 g/L, about 7.2 g/L, about 7.8 g/L, about 8.4 g/L, about 9 g/L, about 10 g/L, about 12 g/L, about 14 g/L, about 16 g/L, about 18 g/L, about 20 g/L, about 25 g/L, about 30 g/L, about 35 g/L, about 40 g/L, about 45 g/L, about 50 g/L, about 55 g/L, or about 60 g/L.

In some embodiments, the one or more stabilizers are one or more surfactants or polymers. In some embodiments, the one or more stabilizers are one or more selected from the group consisting of polyethylene glycol (PEG), polypropylene glycol (PPG), polybutylene glycol (PBG), and polypentylene glycol, cetyltrimethylammonium bromide (CTAB), sodium dodecyl sulfate (SDS), poly(ethylene oxide) (PEO), polyoxyethylene sorbitan monolaurate (Tween®), octyl phenol ethoxylate (Triton™ X-100), polyvinylpyrrolidone (PVP), polyvinyl alcohol (PVA), and combinations thereof. In some embodiments, the polymers are one or more co-polymers. In some embodiments, the one or more co-polymers comprise two or more selected from the group consisting of PEG, PPG, PBG, and polypentylene glycol. In some embodiments, the co-polymer is a block co-polymer. In some embodiments, the block co-polymer is a diblock co-polymer, a triblock co-polymer, a tetrablock co-polymer, or a pentablock co-polymer. In some embodiments, the block co-polymer is triblock co-polymer of PEG and PPG. In some embodiments, the block co-polymer is a PEG-PPG-PEG triblock co-polymer.

In some embodiments, the solution is at between about 25° C. and about 100° C. In some embodiments, the solution is at between about 30° C. and about 80° C. In some embodiments, the solution is at between about 40° C. and about 70° C. In some embodiments, the solutions are at about 25° C., about 30° C., about 40° C., about 50° C., about 60° C., about 70° C., about 80° C., or about 100° C. In some embodiments, the solutions are at different temperatures.

In some embodiments, before contacting the surface with the solution, the solution is microwaved. In some embodiments, the solution is microwaved to between about 30° C. and about 100° C. In some embodiments, the solution is microwaved to between about 40° C. and about 80° C. In some embodiments, the solution is microwaved to between about 50° C. and about 70° C. In some embodiments, the solution is microwaved to about 25° C., about 30° C., about 40° C., about 50° C., about 55° C., about 60° C., about 65° C., about 70° C., about 80° C., or about 100° C.

In some embodiments, the solution is microwaved for between about 1 second and about 30 seconds. In some embodiments, the solution is microwaved for between about 2 seconds and about 15 seconds. In some embodiments, the solution is microwaved for between about 3 seconds and about 10 seconds. In some embodiments, the solution is microwaved for between about 4 seconds and about 6 seconds. In some embodiments, the solution is microwaved for about 1 second, about 2 seconds, about 3 seconds, about 4 seconds, about 5 seconds, about 6 seconds, about 7 seconds, about 8 seconds, about 9 seconds, about 10 seconds, about 15 seconds, about 20 seconds, about 25 seconds, or about 30 seconds.

In some embodiments, after being microwaved and before being contacted with the surface, the solution is held at the temperature achieved for between about 1 minute and about 90 minutes. In some embodiments, the solution is held at the temperature achieved for between about 5 minutes and about 75 minutes. In some embodiments, the solution is held at the temperature achieved for between about 15 minutes and about 60 minutes. In some embodiments, the solution is held at the temperature achieved for between about 20 minutes and about 45 minutes. In some embodiments, the solution is held at the temperature achieved for between about 25 minutes and about 35 minutes. In some embodiments, the solution is held at the temperature achieved for about 1 minute, about 5 minutes, about 10 minutes, about 15 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 45 minutes, about 50 minutes, about 55 minutes, about 60 minutes, about, 75 minutes, or about 90 minutes.

In some embodiments, after being held at a temperature achieved by microwaving, the solution is cooled to room temperature.

In some embodiments, the solution comprising one or more Mn²⁺ salts, one or more MnO₄⁻ salts, and one or more polymers is aqueous. In some embodiments, the solution comprises a combination of water and one or more organic solvents. Examples of suitable organic solvents include, but are not limited to, acetone, ethyl acetate, dimethyl sulfoxide, acetonitrile, and dimethylformamide. In some embodiments, the solutions are in different solvents.

In some embodiments, the surface to be coated with MnO₂ comprises one or more selected from the group consisting of plastic, wood, metal, glass, fabric, stone, or a combination thereof. Examples of plastics suitable for coating include, but are not limited, to polyvinyl chloride (PVC), high-density polyethylene (HDPE), low-density polyethylene (LDPE), polypropylene (PP), polystyrene (PS), polyethylene terephthalate (PET), polyurethane (PUR), polycarbonate (PC), acrylonitrile butadiene styrene (ABS), polysiloxanes, polyetheretherketone (PEEK), polyetherimide (PEI), polyimide, polysulfone, polyamide-imide (PAI), copolymers thereof, and combinations thereof. Examples of metals suitable for coating include, but are not limited to, copper, zinc, iron, titanium, steel, stainless steel, brass, bronze, aluminum, chromium, magnesium alloys, nickel, tin, tungsten, and combinations or alloys thereof.

In some embodiments, the invention provides methods of preparing an iron oxide, lanthanide, or iron oxide-lanthanide composite surface coating. In some embodiments, the method comprises a step of obtaining a surface to be coated and exposing it to a bacterium in the presence of a metal or metal oxide. In some embodiments, the method comprises placing a substrate in a bacterial culture comprising one or more selected from the group consisting of one or more lanthanide salt, one or more iron salts, one or more nanosized lanthanides, one or more nanosized iron, and one or more nanosized iron oxides, and allowing the bacteria to form a composite coating on the surface of the substrate. In some embodiments, the bacteria are acidophiles. In some embodiments, the bacteria are one or more selected from the group consisting of Acidithiobacillus ferrooxidans, Thermoplasma acidophilum, Acidobacterium capsulatum, and Acidimicrobium ferrooxidans. In some embodiments, the bacteria are cultured under aerobic, anaerobic, or facultatively anaerobic conditions. In some embodiments, the bacterially synthesized composite coating comprises nanosized iron, nanosized lanthanides, or nanosized iron-lanthanides composites.

In some embodiments, the bacterial culture comprises a nanosized iron, a nanosized lanthanide, or a combination thereof. In some embodiments, the nanosized iron is a nanosized iron oxide. In some embodiments, the nanosized iron oxide is prepared by a method comprising the steps of a) preparing a solution comprising an iron salt, b) heating the resulting solution, c) cooling the solution, d) centrifuging the solution, and e) collecting the resulting nanosized iron oxide.

In some embodiments, the iron salt of step a) is one or more selected from the group consisting of iron chloride, iron bromide, iron iodide, iron nitrate, iron acetate, and iron acetylacetonate. In some embodiments, the concentration of iron salt in the solution of step a) is between about 1 mg/mL and about 100 mg/mL. In some embodiments, the concentration of iron salt is about 1 mg/mL, about 2 mg/mL, about 3 mg/mL, about 4 mg/mL, about 5 mg/mL, about 6 mg/ml, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, or about 100 mg/mL.

In some embodiments, the solvent in the solution of step a) is one or more selected from the group consisting of water, methanol, ethanol, acetone, dimethylformamide (DMF), dimethyl sulfoxide (DMSO), and N-methyl-2-pyrrolidone (NMP).

In some embodiments, the pH of the solution of step a) determines the size, shape, and phase of the resulting nanosized iron oxide. Accordingly, in some embodiments, the solution further comprises one or more pH modifiers selected from the group consisting of sodium hydroxide, lithium hydroxide, potassium hydroxide, cesium hydroxide, ammonium hydroxide, hydrochloric acid, sulfuric acid, nitric acid, acetic acid, ascorbic acid, Tris buffer, and HEPES buffer. In some embodiments, the concentration of the pH modifier in the solution of step a) is between about 1 mM and about 1 M. In some embodiments, the concentration is about 1 mM, about 2 mM, about 3 mM, about 4 mM, about 5 mM, about 6 mm, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, about 60 mM, about 70 mM, about 80 mM, about 90 mM, about 100 mM, about 150 mM, about 200 mM, about 250 mM, about 300 mM, about 350 mM, about 400 mM, about 450 mM, about 500 mM, about 600 mM, about 700 mM, about 800 mM, about 900 mM, or about 1 M.

In some implementations, the solution further comprises a stabilizer to control the particle size and morphology. In some embodiments, the molar ratio of stabilizer to iron salt is between about 1:1 and about 1:60. In some embodiments, the stabilizer is a polymer or surfactant. Examples of stabilizers include, but are not limited to, cetyltrimethylammonium bromide (CTAB), sodium dodecyl sulfate (SDS), poly(ethylene oxide) (PEO), polyoxyethylene sorbitan monolaurate (Tween®), octyl phenol ethoxylate (Triton™ X-100), polyvinylpyrrolidone (PVP), polyethylene glycol (PEG), polypropylene glycol (PPG), polyvinyl alcohol (PVA), and combinations thereof (e.g., co-polymers).

In some embodiments, step b) comprises heating the solution resulting from step a) in a microwave. In some embodiments, the solution is heated by microwave to a temperature of about 30° C., about 35° C., about 40° C., about 45° C., about 50° C., about 55° C., about 60° C., about 67° C., about 70° C., about 75° C., about 80° C., about 85° C., about 90° C., about 95° C., about or about 100° C.

In some embodiments, step b) comprises microwaving the solution resulting from step a) for between about 1 second and about 30 seconds. In some embodiments, the solution is microwaved for between about 2 seconds and about 15 seconds. In some embodiments, the solution is microwaved for between about 3 seconds and about 10 seconds. In some embodiments, the solution is microwaved for between about 4 seconds and about 6 seconds. In some embodiments, the solution is microwaved for about 1 second, about 2 seconds, about 3 seconds, about 4 seconds, about 5 seconds, about 6 seconds, about 7 seconds, about 8 seconds, about 9 seconds, about 10 seconds, about 15 seconds, about 20 seconds, about 25 seconds, or about 30 seconds.

In some embodiments, step b) further comprises holding the solution at the temperature achieved for between about 1 minute and about 90 minutes. In some embodiments, the solution is held at the temperature achieved for between about 5 minutes and about 75 minutes. In some embodiments, the solution is held at the temperature achieved for between about 15 minutes and about 60 minutes. In some embodiments, the solution is held at the temperature achieved for between about 20 minutes and about 45 minutes. In some embodiments, the solution is held at the temperature achieved for between about 25 minutes and about 35 minutes. In some embodiments, the solution is held at the temperature achieved for about 1 minute, about 5 minutes, about 10 minutes, about 15 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 45 minutes, about 50 minutes, about 55 minutes, about 60 minutes, about, 75 minutes, or about 90 minutes.

In some embodiments, step c) comprises rapidly cooling the solution. In some embodiments, the solution is rapidly cooled to room temperature.

In some embodiments, step d) comprises centrifuging the solution from step c). In some embodiments, the solution is centrifuged at between about 10 rpm and about 10,000×g. In some embodiments, the solution is centrifuged at about 10 rpm, about 20 rpm, about 30 rpm, about 40 rpm, about 50 rpm, about 60 rpm, about 70 rpm, about 80 rpm, about 90 rpm, about 100 rpm, about 125 rpm, about 150 rpm, about 175 rpm, about 200 rpm, about 225 rpm, about 250 rpm, about 300 rpm, about 350 rpm, about 400 rpm, about 450 rpm, about 500 rpm, about 600 rpm, about 700 rpm, about 800 rpm, about 900 rpm, about 1,000 rpm, about 1,100 rpm, about 1,200 rpm, about 1,300 rpm, about 1,400 rpm, about 1,500 rpm, about 2,000 rpm, about 2,500 rpm, about 3,000 rpm, about 3,500 rpm, about 4,000 rpm, about 4,500 rpm, about 5,000 rpm, about 6,000 rpm, about 7,000 rpm, about 8,000 rpm, about 9,000 rpm, or about 10,000 rpm.

In some embodiments, the solution from step c) is centrifuged for between about 1 second and about 1 hour. In some embodiments, the solution is centrifuged for about 1 second, about 2 seconds, about 3 seconds, about 4 seconds, about 5 seconds, about 6 seconds, about 7 seconds, about 8 seconds, about 9 seconds, about 10 seconds, about 15 seconds, about 20 seconds, about 25 seconds, about 30 seconds, about 45 seconds, about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 45 minutes, or about 1 hour.

In some embodiments, step d) further comprises rinsing the nanosized iron oxide obtained by centrifugation one or more times. In some embodiments, the nanosized iron oxide is rinsed one or more times with one or more selected from the group consisting of water, methanol, ethanol, propanol, isopropanol, ethanol, and ethyl acetate.

### MnO₂ Surface Coatings

In various aspects, the present invention provides MnO₂ surface coatings. In some embodiments, the MnO₂ surface coating comprises one or more selected from α-MnO₂, β-MnO₂, and δ-MnO₂. In some embodiments, the MnO₂ surface coating further comprises one or more selected from the group consisting of activated carbon, charcoal, graphene, graphene oxide, graphitic composites, carbon tubes, and carbon fibers. In some embodiments, the activated carbon is granular activated carbon (GAC), powdered activated carbon (PAC), or a combination thereof.

In some embodiments, the MnO₂ surface coating further comprises an adhesive. In some embodiments, the adhesive is one or more selected from the group consisting of water-based polyurethane, oil-based polyurethane, epoxy, two-part epoxy, silica, silicone resin, and combinations thereof.

In some embodiments, an MnO₂ surface coating of the present invention is hydrophobic. In some embodiments, the MnO₂ surface coating is superhydrophobic. In some embodiments, the hydrophobicity of the MnO₂ surface coating is defined by the contact angle of the MnO₂ surface coating and a droplet of water. In some embodiments, the contact angle is between about 1000 and 180°. In some embodiments, the contact angle is between about 1100 and about 180°. In some embodiments, the contact angle is between about 1200 and about 180°. In some embodiments, the contact angle is between about 1300 and about 180°. In some embodiments, the contact angle is about 100°, about 110°, about 120°, about 130°, about 1310, about 132°, about 133°, about 134°, about 135°, or about 136°.

In some embodiments, the thickness of an MnO₂ surface coating of the present invention is between about 0.01 nm and about 1 mm. In some embodiments, the thickness is between about 0.1 nm and about 100 μm. In some embodiments, the thickness is between about 1 nm and about 10 μm. In some embodiments, the thickness is about 0.01 nm, about 0.1 nm, about 1 nm, about 5 nm, about 10 nm, about 15 nm, about 20 nm, about 25 nm, about 50 nm, about 75 nm, about 100 nm, about 125 nm, about 150 nm, about 200 nm, about 300 nm, about 400 nm, about 500 nm, about 600 nm, about 700 nm, about 800 nm, about 900 nm, about 1 μm, about 2 μm, about 3 μm, about 4 μm, about 5 μm, about 10 μm, about 15 μm, about 20 μm, about 30 μm, about 40 μm, about 50 μm, about 60 μm, about 70 μm, about 80 μm, about 90 μm, about 100 μm, about 125 μm, about 150 μm, about 175 μm, about 200 μm, about 300 μm, about 400 am, about 500 μm, about 600 μm, about 700 μm, about 800 μm, about 900 μm, or about 1 mm.

In some embodiments, the MnO₂ surface coating further comprises one or more additional metals. In some embodiments, the one or more additional metals are magnetic, paramagnetic, conductive, or semiconductive. In some embodiments, the one or more additional metals are one or more lanthanides. In some embodiments, the one or more lanthanides are selected from the group consisting of gadolinium, neodymium, erbium, and europium. In some embodiments, the one or more lanthanides are lanthanide oxides. In some embodiments, the metal is iron. In some embodiments, the iron is an iron oxide. In some embodiments, the surface coating comprising one or more metals is produced by biogenic synthesis. In some embodiments, the surface coating is produced by bacteria.

In some embodiments, an MnO₂ surface coating of the present invention further comprises one or more catalysts. Examples of catalysts include, but are not limited to, transition metals, basic metals, semimetals, lanthanides, and actinides. Examples include, but are not limited to, platinum, palladium, ruthenium, rhodium, copper, nickel, and vanadium.

### Magnetic Iron Oxide Surface Coatings:

In various aspects, the present invention provides magnetic iron oxide surface coatings. In some embodiments, the surface coating further comprises one or more selected from the group consisting of activated carbon, charcoal, graphene, graphene oxide, graphitic composites, carbon tubes, and carbon fibers. In some embodiments, the activated carbon is granular activated carbon (GAC), powdered activated carbon (PAC), or a combination thereof. In some embodiments, the surface coating further comprises one or more lanthanides. Examples of lanthanides include, but are not limited to, europium, erbium, gadolinium, neodymium, and lanthanum.

In some embodiments, the thickness of an iron oxide surface coating of the present invention is between about 0.01 nm and about 1 mm. In some embodiments, the thickness is between about 0.1 nm and about 100 μm. In some embodiments, the thickness is between about 1 nm and about 10 μm. In some embodiments, the thickness is about 0.01 nm, about 0.1 nm, about 1 nm, about 5 nm, about 10 nm, about 15 nm, about 20 nm, about 25 nm, about 50 nm, about 75 nm, about 100 nm, about 125 nm, about 150 nm, about 200 nm, about 300 nm, about 400 nm, about 500 nm, about 600 nm, about 700 nm, about 800 nm, about 900 nm, about 1 μm, about 2 μm, about 3 μm, about 4 μm, about 5 μm, about 10 μm, about 15 μm, about 20 μm, about 30 μm, about 40 μm, about 50 μm, about 60 μm, about 70 μm, about 80 μm, about 90 μm, about 100 μm, about 125 μm, about 150 μm, about 175 μm, about 200 μm, about 300 am, about 400 μm, about 500 μm, about 600 μm, about 700 μm, about 800 μm, about 900 μm, or about 1 mm.

In some embodiments, an iron oxide surface coating of the present invention further comprises one or more catalysts. Examples of catalysts include, but are not limited to, transition metals, basic metals, semimetals, lanthanides, and actinides. Examples include, but are not limited to, platinum, palladium, ruthenium, rhodium, copper, nickel, and vanadium.

### Lanthanide Surface Coatings

In various aspects, the present invention provides lanthanide surface coatings. Examples of lanthanides that can be incorporated in the surface coatings include, but are not limited to europium, erbium, gadolinium, neodymium, and lanthanum.

In some embodiments, the surface coating further comprises one or more selected from the group consisting of activated carbon, charcoal, graphene, graphene oxide, graphitic composites, carbon tubes, and carbon fibers. In some embodiments, the activated carbon is granular activated carbon (GAC), powdered activated carbon (PAC), or a combination thereof.

In some embodiments, the thickness of a lanthanide surface coating of the present invention is between about 0.01 nm and about 1 mm. In some embodiments, the thickness is between about 0.1 nm and about 100 μm. In some embodiments, the thickness is between about 1 nm and about 10 μm. In some embodiments, the thickness is about 0.01 nm, about 0.1 nm, about 1 nm, about 5 nm, about 10 nm, about 15 nm, about 20 nm, about 25 nm, about 50 nm, about 75 nm, about 100 nm, about 125 nm, about 150 nm, about 200 nm, about 300 nm, about 400 nm, about 500 nm, about 600 nm, about 700 nm, about 800 nm, about 900 nm, about 1 μm, about 2 μm, about 3 μm, about 4 μm, about 5 μm, about 10 μm, about 15 μm, about 20 μm, about 30 μm, about 40 μm, about 50 μm, about 60 μm, about 70 μm, about 80 μm, about 90 μm, about 100 μm, about 125 μm, about 150 μm, about 175 μm, about 200 μm, about 300 μm, about 400 am, about 500 μm, about 600 μm, about 700 μm, about 800 μm, about 900 μm, or about 1 mm.

In some embodiments, a lanthanide surface coating of the present invention further comprises one or more catalysts. Examples of catalysts include, but are not limited to, transition metals, basic metals, semimetals, lanthanides, and actinides. Examples include, but are not limited to, platinum, palladium, ruthenium, rhodium, copper, nickel, and vanadium.

### Plug Flow Reactors

In various aspects, the present invention provides improved plug flow reactors (PFRs). In some embodiments, the PFR comprises a plurality of pipes, tubes, and/or lines which are partially or fully coated by a surface coating of the present invention according to any of the methods of the present invention. In some embodiments, between about 1% and about 100% of the interior surfaces of the pipes, tubes, and/or lines are coated with a surface coating. In some embodiments, at least 1%, at least 2%, at least 3%, at least 4%, at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, at least 99.5%, at least 99.6%, at least 99.7%, at least 99.8%, at least 99.9%, or at least 99.99% of the interior surfaces of the pipes, tubes, and/or lines are coated with a surface coating.

In some embodiments, the PFR comprises a static flow mixer, wherein the surface of the static flow mixer. In some embodiments, the surface of the static flow mixer is fully or partially coated with a surface coating. In some embodiments, between about 1% and about 100% of the surface of the static flow mixer is covered with a surface coating. In some embodiments, at least 1%, at least 2%, at least 3%, at least 4%, at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, at least 99.5%, at least 99.6%, at least 99.7%, at least 99.8%, at least 99.9%, or at least 99.99% of the surface of the static flow mixer is coated with a surface coating.

In some embodiments, the PFR comprises a static flow mixer, both the interior surfaces of the pipes, tubes, and/or lines of the PFR and the surface of the static flow mixer are fully or partially coated with a surface coating of the present invention. In some embodiments, between about 1% and about 100% of the interior surfaces of the pipes, tubes, and/or lines are coated with a surface coating. In some embodiments, at least 1%, at least 2%, at least 3%, at least 4%, at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, at least 99.5%, at least 99.6%, at least 99.7%, at least 99.8%, at least 99.9%, or at least 99.99% of the interior surfaces of the pipes, tubes, and/or lines are coated with a surface coating. In some embodiments, between about 1% and about 100% of the surface of the static flow mixer is covered with an MnO₂ coating. In some embodiments, at least 1%, at least 2%, at least 3%, at least 4%, at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, at least 99.5%, at least 99.6%, at least 99.7%, at least 99.8%, at least 99.9%, or at least 99.99% of the surface of the static flow mixer is coated with a surface coating.

### Dialysis Lines

In various aspects, the present invention provides improved dialysis lines. In some embodiments, the dialysis line comprises an adsorbent layer coating the interior of the line. In some embodiments, the adsorbent layer is any surface coating of the present invention prepared by any method of the present invention. In some embodiments, the adsorbent layer comprises activated carbon. In some embodiments, the adsorbent layer comprises an absorptive polymer. Examples of absorptive polymers include, but are not limited to, sevelamer, polyacrylates, polystyrene (PS), polyethylene glycol (PEG), polyvinyl alcohol (PVA), polyallylamines, polyvinylpyrrolidone (PVP), polyurethanes, polyethylenimine (PEI), chitosan, poly(methyl methacrylate) (PMMA), poly(methacrylic acid) (PMAA), and combinations thereof.

In some embodiments, the adsorbent layer has a thickness between about 0.01 nm and about 1 mm. In some embodiments, the thickness is between about 0.1 nm and about 100 m. In some embodiments, the thickness is between about 1 nm and about 10 μm. In some embodiments, the thickness is about 0.01 nm, about 0.1 nm, about 1 nm, about 5 nm, about 10 nm, about 15 nm, about 20 nm, about 25 nm, about 50 nm, about 75 nm, about 100 nm, about 125 nm, about 150 nm, about 200 nm, about 300 nm, about 400 nm, about 500 nm, about 600 nm, about 700 nm, about 800 nm, about 900 nm, about 1 μm, about 2 μm, about 3 μm, about 4 m, about 5 μm, about 10 μm, about 15 μm, about 20 μm, about 30 μm, about 40 μm, about 50 am, about 60 μm, about 70 μm, about 80 μm, about 90 μm, about 100 μm, about 125 μm, about 150 μm, about 175 μm, about 200 μm, about 300 μm, about 400 μm, about 500 μm, about 600 am, about 700 μm, about 800 μm, about 900 μm, or about 1 mm.

In some embodiments, the adsorbent layer is pre-impregnated with one or more additives that slowly release into fluid as it passes over the layer. Examples of additives include, but are not limited to, vitamins, minerals, ions, amino acids, analgesics, anesthetics, antibiotics, antivirals, anti-helminthics, anti-fungals, anticoagulants, and anti-cancer agents.

In some embodiments, the line further comprises a porous layer covering the exposed surface of the adsorbent layer. In some embodiments, the porous layer allows fluid access to the adsorbent layer while preventing cellular contact with the adsorbent layer. In some embodiments, the porous layer comprises a biocompatible polymer. In some embodiments, the biocompatible polymer is one or more selected from the group consisting of polystyrene, polypropylene, polyvinylchloride, polyethylene, polyurethane, polycarbonate, polyethylene terephthalate, polyether ether ketone, PEG, PPG, polysulfone (PSU), polyethersulfone (PES), polyacrylonitrile (PAN), polyamide, cellulose acetate, modified cellulose (e.g., hemophan), polyvinylpyrrolidone (PVP), poly(methyl methacrylate) (PMMA), polyethylene glycol diacrylate, poly(lactic-co-glycolic acid) (PLGA), polyvinyl alcohol (PVA), polyacrylamide, polytetrafluoroethylene, chitosan, polypropylene glycol (PPG), and combinations thereof. In some embodiments, the polymer comprises an anticoagulant. In some embodiments, the anticoagulant is heparin.

In some embodiments, the porous layer has a thickness between about 0.01 nm and about 1 mm. In some embodiments, the thickness is between about 0.1 nm and about 100 m. In some embodiments, the thickness is between about 1 nm and about 10 μm. In some embodiments, the thickness is about 0.01 nm, about 0.1 nm, about 1 nm, about 5 nm, about 10 nm, about 15 nm, about 20 nm, about 25 nm, about 50 nm, about 75 nm, about 100 nm, about 125 nm, about 150 nm, about 200 nm, about 300 nm, about 400 nm, about 500 nm, about 600 nm, about 700 nm, about 800 nm, about 900 nm, about 1 μm, about 2 μm, about 3 μm, about 4 m, about 5 μm, about 10 μm, about 15 μm, about 20 μm, about 30 μm, about 40 μm, about 50 am, about 60 μm, about 70 μm, about 80 μm, about 90 μm, about 100 μm, about 125 μm, about 150 μm, about 175 μm, about 200 μm, about 300 μm, about 400 μm, about 500 μm, about 600 am, about 700 μm, about 800 μm, about 900 μm, or about 1 mm.

In some embodiments, the porous layer has an average pore diameter between about 0.01 nm and about 6 μm. In some embodiments, the average pore diameter is between about 0.1 nm and about 6 μm. In some embodiments, then average pore diameter is between about 1 nm and about 6 μm. In some embodiments, the average pore diameter is between about 10 nm and about 6 μm. In some embodiments, the average pore diameter is between about 100 nm and about 6 μm. In some embodiments, the average pore diameter is about 0.01 nm, about 0.1 nm, about 1 nm, about 10 nm, about 20 nm, about 30 nm, about 40 nm, about 50 nm, about 60 nm, about 70 nm, about 80 nm, about 90 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1 μm, about 1.5 μm, about 2 μm, about 2.5 μm, about 3 μm, about 3.5 μm, about 4 μm, about 4.5 μm, about 5 μm, about 5.5 μm, or about 6 μm.

In some embodiments, the dialysis line has an inner diameter between about 0.5 mm and about 15 mm. In some embodiments, the inner diameter is between about 1 mm and about 10 mm. In some embodiments, the inner diameter is between about 2 mm and about 8 mm. In some embodiments, the inner diameter is about 0.5 mm, about 1 mm, about 1.5 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 11 mm, about 12 mm, about 13 mm, about 14 mm, or about 15 mm.

In some embodiments, the dialysis line has an effective inner diameter (the diameter of the hollow space within the tube not occupied by the adsorbent layer or the biocompatible porous layer) between about 0.5 mm and about 15 mm. In some embodiments, the effective inner diameter is between about 1 mm and about 10 mm. In some embodiments, the effective inner diameter is between about 2 mm and about 8 mm. In some embodiments, the effective inner diameter is about 0.5 mm, about 1 mm, about 1.5 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 11 mm, about 12 mm, about 13 mm, about 14 mm, or about 15 mm.

### Methods of Inhibiting Bacteria

In a variety of industries, including food and beverage and pharmaceuticals, sterility of fluids is critical. Accordingly, in various aspects, the present invention provides methods of inhibiting bacteria on a surface. In one embodiment, the method comprises treating the surface according to any method of coating a surface with surface coating of the present invention.

In some embodiments, the method is useful for preventing the growth of bacteria on a surface or killing bacteria that come in contact with a surface. In some embodiments, the method is useful for disrupting, destroying, reducing the rate of growth of, or preventing the formation of bacterial biofilms. In some embodiments, the reduced rate of growth of a biofilm is determined as compared to the rate of growth of a biofilm on the same surface without a coating of a surface coating of the present invention.

In some embodiments, the method is useful for inhibiting Gram-positive and/or Gram-negative bacteria. In some embodiments, the method is useful for killing Gram-positive and/or negative bacteria.

In some embodiments, the method comprises passing a fluid with bacteria in it over a surface coated with a surface coating according to any of the methods of the present invention. In some embodiments, the surface coated with a surface coating is an interior surface of a plug flow reactor (PFR). In some embodiments, the inner walls, or a portion thereof, of the pipes, lines, and/or tubes of a PFR are coated with a surface coating. In some embodiments, the surface of one or more static flow mixers within a PFR are coated with a surface coating.

Examples of fluids which may be used in a method of the invention include, but are not limited to, water, reaction solvents, culture media, blood, serum, plasma, spinal fluid, urine, dialysis solution, wastewater, liquid foods (e.g., syrups, marinades, brines, etc.), beverages (e.g., milk, juice, alcoholic beverages, etc.), oils, and liquid fats.

### Methods of Catalysis:

Catalysis is essential in a wide variety of industries. In pharmaceutical manufacturing catalysis is used for synthesizing drug compounds efficiently and selectively. Catalysts facilitate key chemical reactions, such as hydrogenation and oxidation, crucial for creating complex molecular structures in drugs. They are particularly vital in asymmetric synthesis for producing chiral molecules, directly impacting the therapeutic effectiveness of many pharmaceuticals.

In the petrochemical industry, fluid purification is a vital process for refining crude oil and gas into various products. This involves removing impurities, such as sulfur, nitrogen compounds, and heavy metals, using methods like hydrodesulfurization, adsorption, and catalytic cracking. Catalysis is critical for refining crude oil and gas, prominently in processes like fluid catalytic cracking (FCC) and hydrocracking. These catalyst-driven reactions are essential for converting complex hydrocarbons into simpler, valuable products like gasoline and diesel. Additionally, catalysis facilitates reactions like olefin polymerization and alkylation, crucial for chemical and polymer production.

Accordingly, in various aspects, the present invention provides methods of catalyzing reactions. In some embodiments, the method comprises the step of passing a fluid in need of catalysis over a surface coated with a surface coating of the present invention. In some embodiments, the surface coated with the surface coating is the interior surfaces of a pipe in a plug flow reactor (PFR). In some embodiments, the surface coated with the surface coating is a static mixer in a plug flow reactor.

### Methods of Removing Contaminants

In various industries such as pharmaceuticals, petrochemicals, and food and beverages, fluid purification through adsorption plays a crucial role. In the pharmaceutical industry, adsorption, primarily using activated carbon or specialized resins, is essential for removing impurities and contaminants from solvents, Water for Injection (WFI), and process fluids, to ensure product safety and efficacy. In the petrochemical industry, adsorption is employed for the purification of hydrocarbon streams, removing sulfur compounds and other impurities to meet product and environmental standards. In the food and beverage sector, adsorption techniques are used to refine oils, water, and other liquids, effectively eliminating undesired components such as colors, odors, and flavors, thereby ensuring product quality and compliance with safety regulations.

In hemodialysis for patients with chronic kidney disease (CKD) and end-stage renal disease (ESRD), the efficient removal of small molecules and protein-bound uremic toxins is critical. Their removal is essential to maintain electrolyte balance and prevent complications such as hyperkalemia.

Accordingly, in various aspects, the present invention provides methods of removing contaminants from a fluid. In one embodiment, the method comprises contacting a surface coated with a surface coating according to any method of the present invention with a fluid contaminated with one or more contaminants. In some embodiments, the one or more contaminants are one or more heavy metals, one or more hydrophobic small molecules, one or more peptides or proteins, and one or more molecules or ions that are normally present in the fluid but are in the fluid at an elevated or unhealthy level. In some embodiments, the surface adsorbs heavy metals from the aqueous environment.

Examples of heavy metals which may be removed from a fluid include, but are not limited to, chromium, arsenic, cadmium, mercury, lead, vanadium, osmium, platinum, indium, bismuth, uranium, antimony, and thallium.

Examples of ions which may be removed from a fluid include but are not limited to, calcium, copper, magnesium, aluminum, iron, potassium, sodium, chloride, sulfate, nitrate, phosphate, carbonate, bicarbonate, fluoride, bromide, iodide, cyanide, sulfide, perchlorate, arsenate, and borate.

Examples of hydrophobic small molecules which may be removed from a fluid include, but are not limited to, alkyl, alkenyl, alkynl, haloalkyl, haloalkenyl, haloalkynyl, heteroalkyl, heteroalkenyl, heteroalkynyl, cycloalkyl, cycloalkenyl, cylcoalkynl, heterocycloalkyl, heterocycloalkenyl, heterocylcoalkynyl, aryl, and heteroaryl compounds, and combinations thereof.

Examples of molecules that are normally present in the fluid but are in the fluid at an elevated level that may be removed from the fluid include, but are not limited to, glucose, sucrose, phosphate, chloride, sodium, potassium, indoxyl sulfate, p-cresyl sulfate, hippuric acid, trimethylamine N-oxide (TMAO), and asymmetric dimethylarginine (ADMA), creatinine, urea, indole-3-acetic acid, phenylacetylglutamine, homocysteine.

Additionally, elevated levels of fibroblast growth factor 23 (FGF23), a hormone involved in phosphate regulation, are also observed in these patients. The high affinity of these toxins for plasma proteins makes their removal during standard dialysis challenging, highlighting the need for advanced techniques for effective clearance.

Examples of proteins and peptides that may be removed from a fluid include, but are not limited to interleukin 6 (IL-6), tumor necrosis factor α (TNF-α), fibroblast growth factor 23 (FGF23), beta-2-microglobulin, parathyroid hormone, and advanced glycation end products (AGEs).

Examples of fluids from which one or more contaminants may be removed include, but are not limited to, water, reaction solvents, culture media, blood, serum, plasma, spinal fluid, urine, dialysis solution, wastewater, liquid foods (e.g., syrups, marinades, brines, etc.), beverages (e.g., milk, juice, alcoholic beverages, etc.), oils, and liquid fats.

In some embodiments, the method of removing one or more contaminants from a fluid comprises passing the fluid through a PFR, wherein the inner walls, or a portion thereof, of the pipes, lines, or tubes of the PFR are coated with a surface coating according to any of the methods of the present invention. In some embodiments, the PFR further comprises a static flow mixer which is coated with a surface coating according to any of the methods of the present invention.

In some embodiments, the method of removing one or more contaminants from a fluid comprises passing the fluid through a tube coated with a surface coating according to any of the methods of the present invention. In some embodiments, the tube is a dialysis tube. In some embodiments, the tube further comprises a layer of a porous biocompatible polymer. In some embodiments, the porous biocompatible polymer allows the fluid and contaminants contained therein to contact the surface coating layer while preventing cells in the fluid from contacting the surface coating layer.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Uniform Coating of Surfaces with MnO₂

### Dip Method

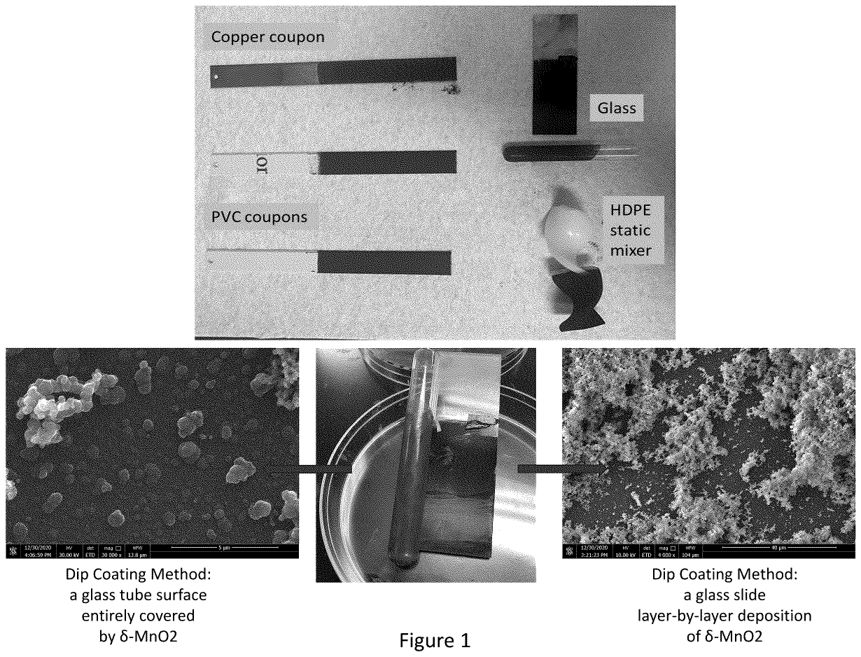

In order to develop a facile method of forming a uniformly layer of δ-MnO₂ of a desired thickness, a method of repeated layering was developed. An aqueous solution of manganese (II) nitrate (Mn(NO₃)₂) was prepared by dissolving 20 g of Mn(NO₃)₂ in 1 L of water at 50° C. An aqueous solution of potassium permanganate (KMnO₄) was prepared by dissolving 20 g of KMnO₄ in 1 L of water at 50° C. A variety of different types of substrates were periodically contacted in the solutions at 50° C. Substrates tested included polyvinyl chloride (PVC) pipes and coupons, copper pipes and coupons, glass tubes and coupons, a high-density polyethylene (HDPE) coupon, and an HDPE static mixer (FIG. 1). The process was repeated several times to form a thin layer of MnO₂ on the surface of the substrate, the thickness of which was controlled by the number of repetitions. The formed layers were then washed with distilled water.

In additional experiments, static mixers were first coated with activated carbon before the MnO₂ layer was formed.

### Microwave Method

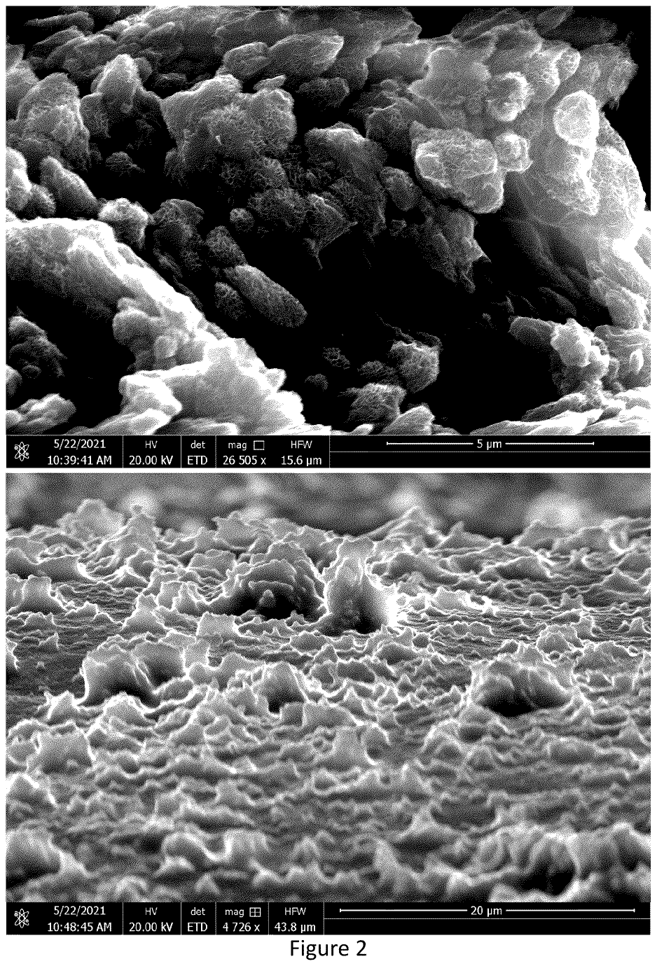

In order to take advantage of the nano-knife effect, as well as to reduce the possibility of air bubbles being trapped in the coating, a microwave synthesis was developed for preparing MnO₂ An aqueous polymer solution of manganese was prepared by dissolving 150 mg of Mn(NO₃)₂ with 60 mg of a polyethylene glycol (PEG)/polypropylene glycol (PPG) triblock copolymer (PEG-PPG-PEG) in 10 mL of distilled water. The solution was mixed with 10 mL of 0.1 M KMnO₄ and microwaved until the solution was at 60° C., at which point the solution was held at that temperature for 30 minutes. The solution was then cooled to room temperature and a variety of substrates were periodically contacted with the solution (FIG. 2).

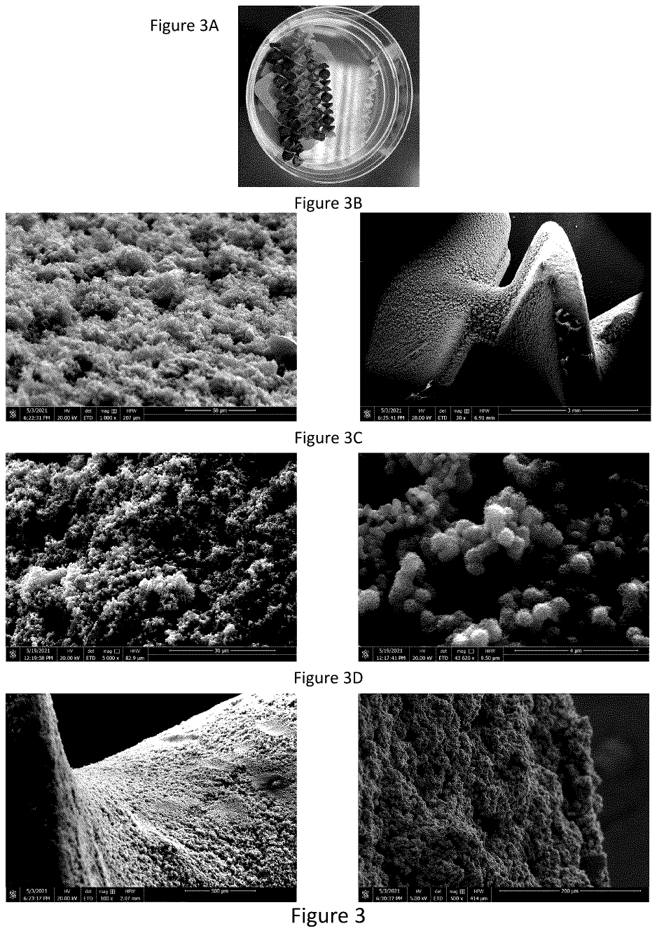

In additional experiments, static mixers were first coated with powdered or granulated activated carbon (PAC or GAC, respectively) before the MnO₂ layer was prepared (FIG. 3).

### Experiment 2: Removal of Lead from an Aqueous Solution

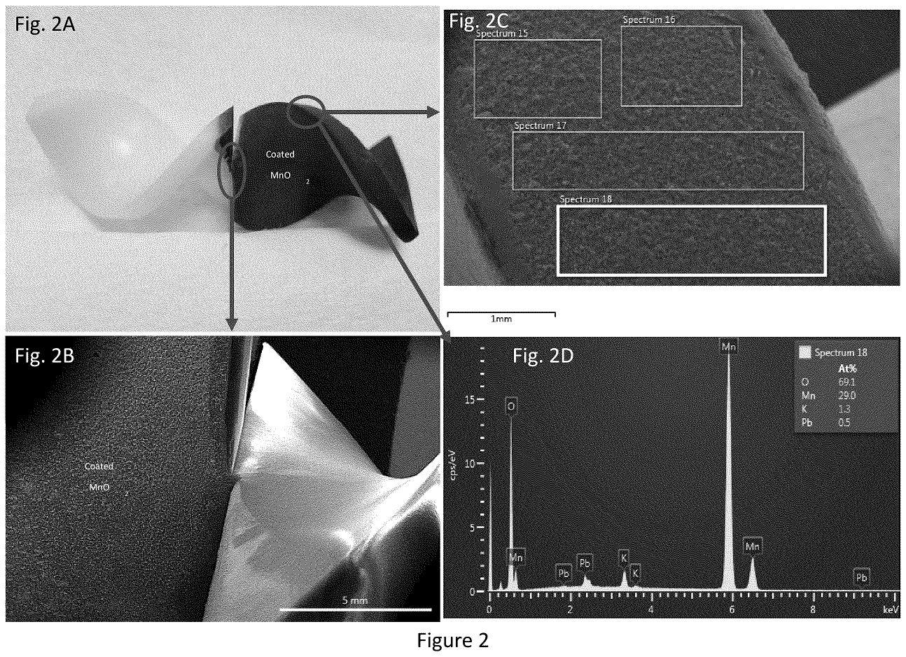

In order to test the absorptive properties of surfaces coated with MnO₂, a static HDPE mixer was coated with MnO₂ according to the previously developed method (FIGS. 4A-4C). The mixer was submerged in water with a lead concentration of 5.6 mg/L and rotated overnight. Measurement of lead levels in the water the following day demonstrated the concentration was reduced to 0.76 mg/L, an 86% reduction. Adsorption of the lead by the coating was confirmed by energy-dispersive X-ray spectroscopy (EDS) analysis (FIG. 4D).

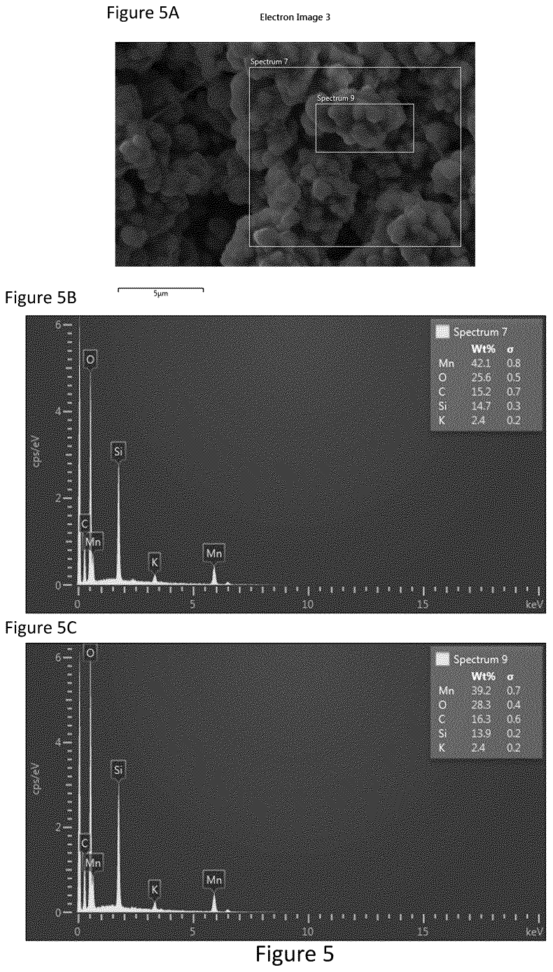

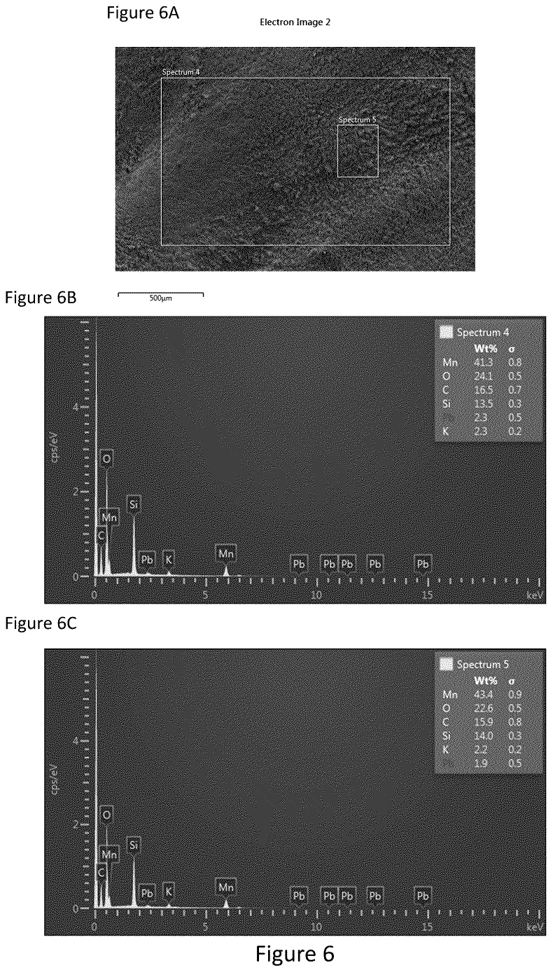

The absorptive properties of an MnO₂-silica composite coating was also examined. Elemental analysis was performed on an MnO₂-silica coated HDPE mixer by EDS demonstrated the presence of carbon, silica, manganese, and potassium, all of which were retained upon exposure of the coated mixer to aqueous lead (FIGS. 5 and 6).

### Experiment 3: Inhibition of Bacterial Growth

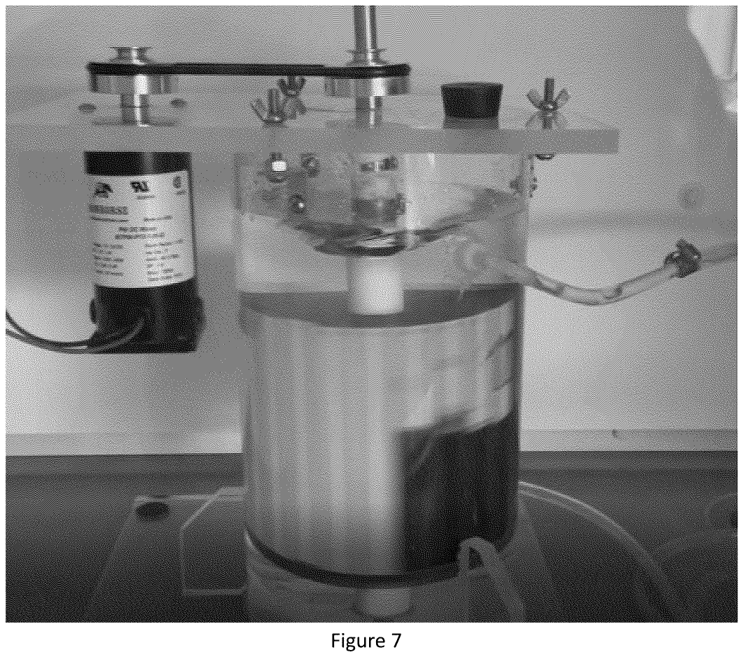

The antibacterial properties of the MnO₂ coating and its impact on biofilm formation was next examined. Copper and PVC coupons were left uncoated as controls or coated with MnO₂ as above and set up in rotating cylinder bioreactors (FIG. 7). After incubation with stirring, samples were taken from each of the bioreactors. Half of each sample was subjected to 16S rRNA qPCR for determination of total cell count while the other half was treated with propidium monoazide (PMA). PMA is a dye used in conjunction with qPCR to differentiate between live and dead cells. PMA penetrates the compromised membranes of dead cells, but not the membranes of live cells. Once inside the cell, PMA intercalates with DNA and, upon exposure to light, covalently bonds to the DNA, inhibiting PCR amplification of the DNA. Accordingly, comparison with total qPCR results provides determination of the proportion of cells killed. Results of the experiment are shown in Table 1.

TABLE 1
Bacterial Viability With and Without MnO₂ Coating

| | | Bacteria | Log₁₀ 16S | | |
|---|---|---|---|---|---|
| Material | Coating | Counted | rRNA | S.D. | % Dead* |
| Copper | Control | Total | 5.22 | 0.02 | 62.8 |
| | | Viable | 4.79 | 0.04 | |
| | MnO₂ | Total | 4.49 | 0.02 | 64.5 |
| | | Viable | 4.04 | 0.02 | |
| PVC | Control | Total | 5.12 | 0.06 | 77 |
| | | Viable | 4.48 | 0.05 | |
| | MnO₂ | Total | 4.49 | 0.02 | 50 |
| | | Viable | 4.19 | 0.04 | |

*Calculated as (16S rRNAₜₒₜₐₗ − 16S rRNA_{viable})/(16S rRNAₜₒₜₐₗ) × 100%

While no significant impact was seen in copper substrates, possibly due to innate antibacterial activity of copper, a significant increase in bacterial cell killing was observed upon addition of an MnO₂ coating to PVC, with cell viability reduced by half.

### Example 4: Superhydrophobicity of MnO₂-Coated Surfaces

Hydrophobicity and hydrophilicity are surface characteristics that significantly influence the adsorption and adsorption processes of contaminants in water. Hydrophobic contaminants tend to be adsorbed onto hydrophobic surfaces, while hydrophilic contaminants prefer hydrophilic surfaces, or remaining in solution. The extent of adsorption and absorption is determined by the compatibility of the surface properties with the characteristics of a molecular contaminant. For instance, while a hydrophobic surface has low affinity for water and a high affinity for other hydrophobic molecules, leading to hydrophobic contaminants being more likely to be adsorbed onto the surface. Hydrophobic surfaces tend not to absorb water or hydrophilic contaminants because the process of absorption involves the penetration of molecules into the hydrophilic substrate, when molecular interactions with water would favor the molecule remaining in solution while hydrophobic molecules will more readily be absorbed.

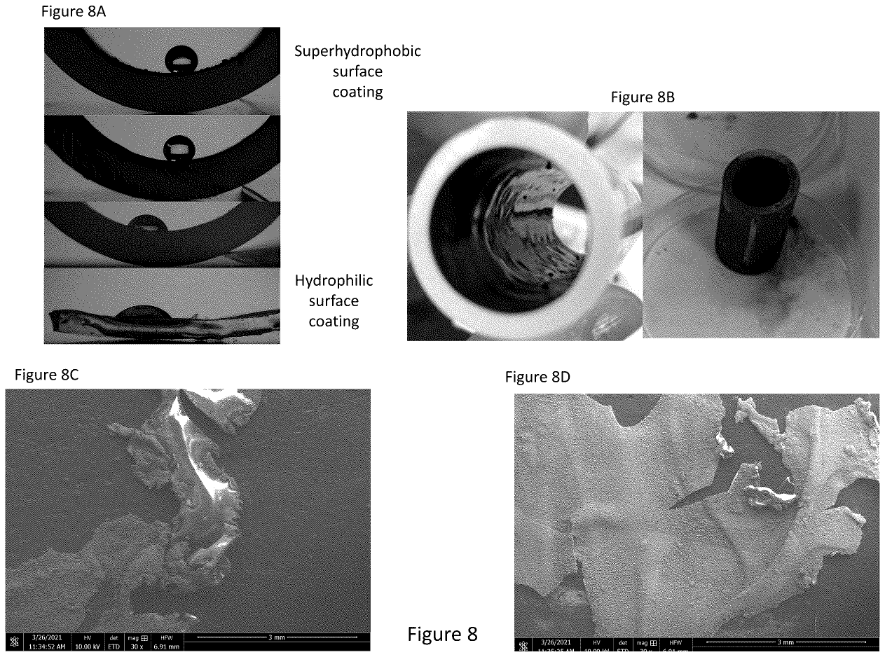

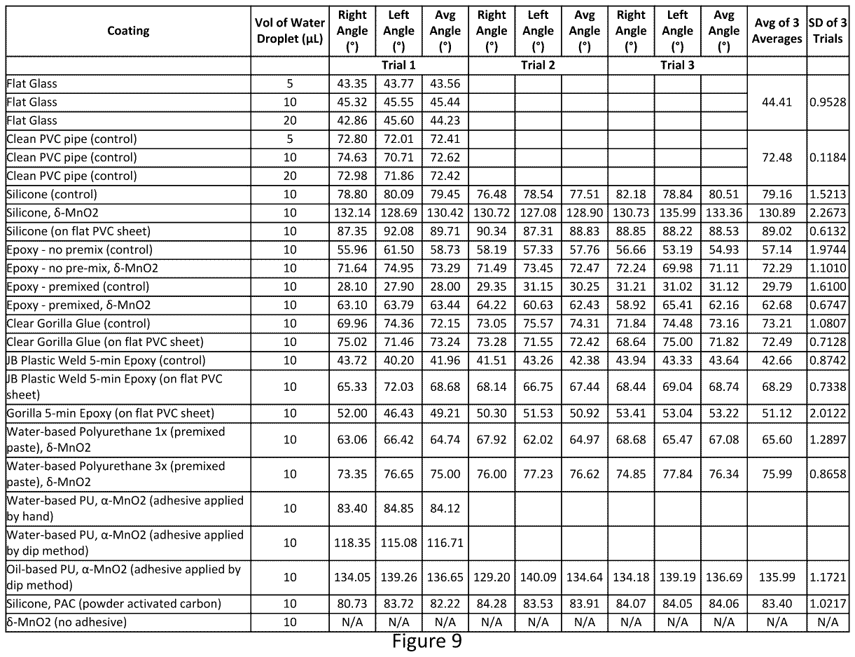

In order to test the hydrophobicity/hydrophilicity of the MnO₂ coatings, an MnO₂ coating and adhesive-MnO₂ combination coatings were examined by their contact angle with water (FIG. 8). In investigating the combination of MnO₂ with adhesives, several representative adhesives were chosen: a silica epoxy, Clear Gorilla Glue® (polyurethane prepolymer), a water-based polyurethane, and J-B PlasticWeld® (two-part epoxy-amine adhesive). PVC pipes were utilized as a substrate left untreated (control) or treated with various mixtures of adhesives and MnO₂ (Table 2, FIG. 9). The nanomaterial coatings with resins and polymers are applied to the surface by spin coating, dip coating, spray coating, layer-by-layer assembly, chemical vapor deposition, electrophoretic deposition, electrospinning, and sol-gel processing.

TABLE 2
Contact Angle of Coatings

| | Vol. | Contact | |
|---|---|---|---|
| | Water | Angle | |
| Coating | Drop (μL) | (Avg.) | SD |
| Flat Glass | 5 | 43.56 | 0.95 |
| | 10 | 45.44 | |
| | 20 | 44.23 | |
| No Coating | 5 | 72.41 | 0.12 |
| | 10 | 72.62 | |
| | 20 | 72.42 | |
| Silica Epoxy | 10 | 79.16 | 1.52 |
| Silica Epoxy + MnO₂ | 10 | 130.89 | 2.27 |
| Silica Epoxy (on Flat PVC) | 10 | 89.02 | 0.61 |
| Epoxy Resin − No Premix | 10 | 57.14 | 1.97 |
| Epoxy Resin − No Premix + MnO₂ | 10 | 72.29 | 1.10 |
| Epoxy Resin − Premixed | 10 | 29.79 | 1.61 |
| Epoxy Resin − Premixed + MnO₂ | 10 | 62.68 | 0.67 |
| Clear Gorilla ® Glue | 10 | 73.21 | 1.08 |
| Clear Gorilla ® Glue (on flat PVC) | 10 | 72.49 | 0.71 |
| J-B PlasticWeld ® | 10 | 42.66 | 0.87 |
| J-B PlasticWeld ® (on flat PVC) | 10 | 68.29 | 0.73 |
| Gorilla ® Epoxy (on flat PVC) | 10 | 51.12 | 2.01 |
| Water-based PU | 10 | 65.60 | 1.29 |
| (1×, premixed) + MnO₂ | | | |
| Water-based PU | 10 | 75.99 | 0.87 |
| (3×, premixed) + MnO₂ | | | |
| Water-based PU + MnO₂ | 10 | * | N/A |
| (Adhesive applied by hand). | | | |
| Water-based PU + MnO₂ | 10 | * | N/A |
| (Adhesive applied by dip method) | | | |
| Oil-based PU + MnO₂ | 10 | 135.99 | 1.17 |
| (adhesive applied by dip method) | | | |
| Silica Epoxy + PAC | 10 | 83.40 | 1.02 |
| MnO₂ | 10 | † | N/A |

*, a bubble was initially observed, but flattened within one minute, not allowing measurement of the angle;
†, no bubble formed; N/A no SD was calculated.

### Example 5: Application to Plug Flow Reactors

Plug flow reactors (PFRs) have become important tools for industrial scale chemical, biochemical, and biological applications. A benefit of PFRs is the minimal use of moving parts, resulting from the incorporation of static mixers, which take advantage of fluid flow to continuously mix the fluid without active stirring. However, as PFR systems utilize stationary parts, they become susceptible to biofilm formation. To mitigate this, the interior walls of a plug flow reactor, as well as the entire surface of the static mixer, are coated with an MnO₂ coating.

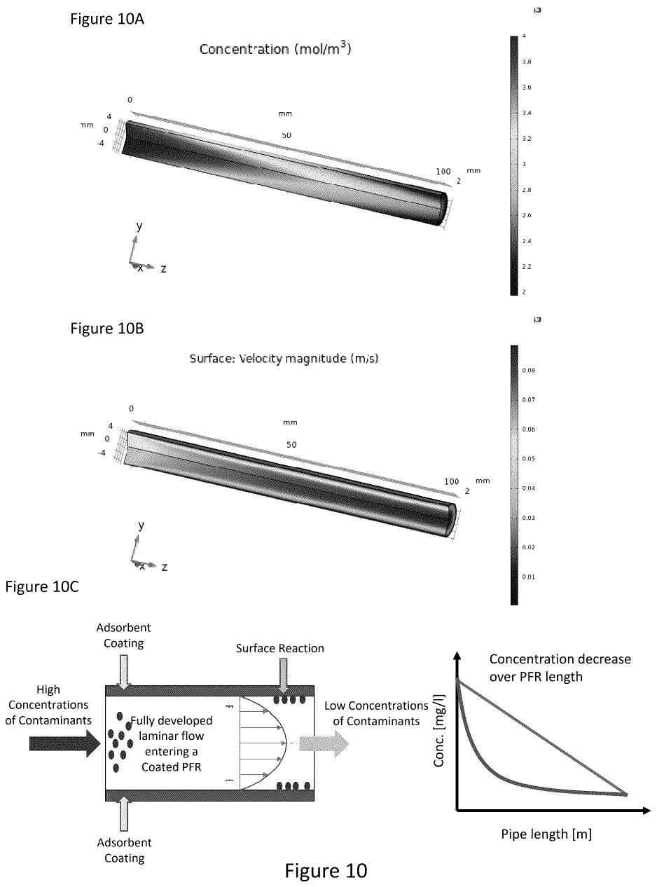

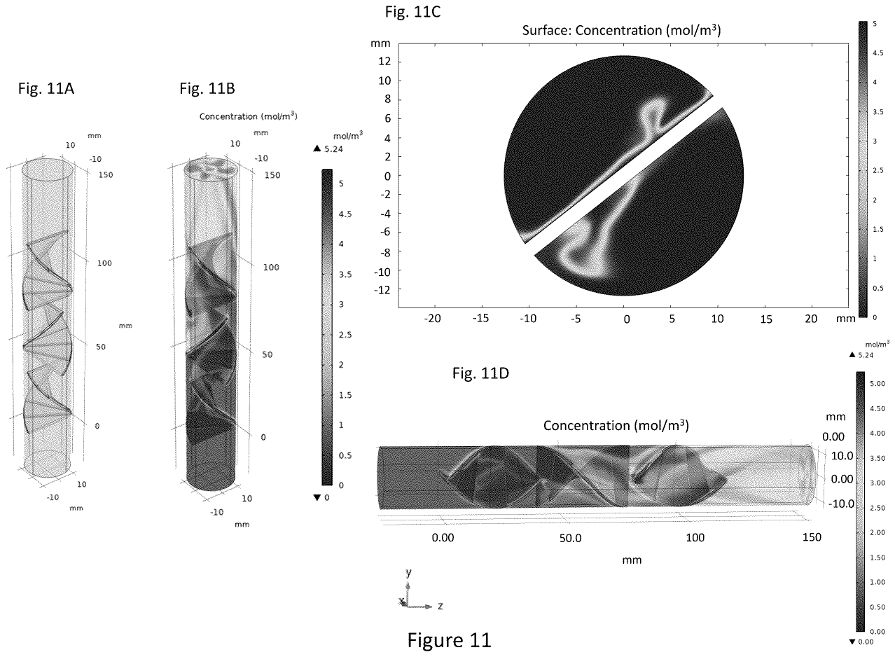

Additionally, a benefit of the MnO₂ coating is the ability to remove contaminants from the fluid flowing through the PFR. Coating of the interior walls and static mixer allows for adsorption of contaminants, such as lead, from the fluid. Modeling of lead removal in a PFR without a static mixer is depicted in FIG. 10. By comparison, modeling of lead removal in a PFR with a static mixer demonstrates a significant reduction in lead concentration over a significantly shorter distance than is required without a static mixer (FIG. 11).

The superhydrophobicity of MnO₂-adhesive coatings further improves adsorption of hydrophobic contaminants while reducing fluid stagnation along PFR walls and ensuring a more uniform flow velocity.

### Example 6: Application to Hemodialysis and Hemoperfusion

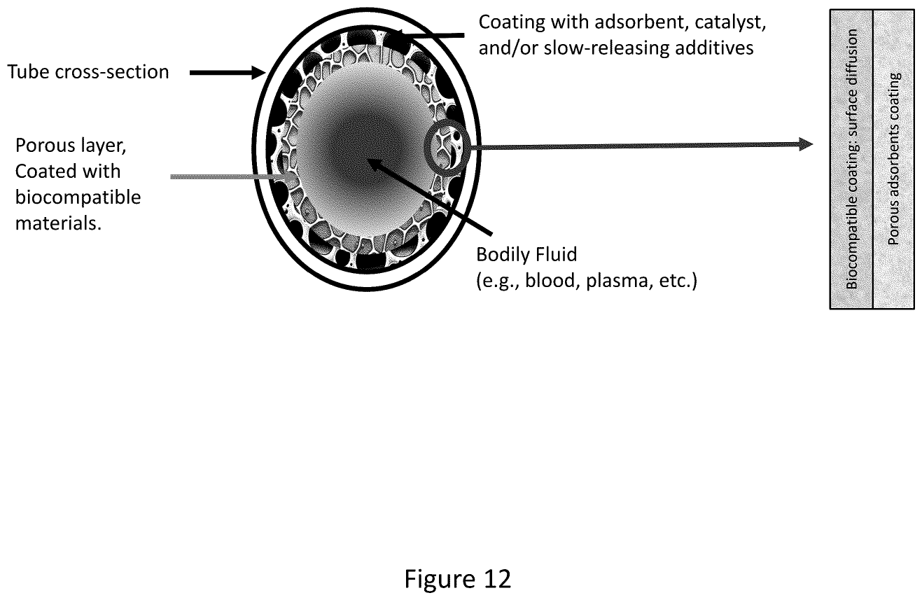

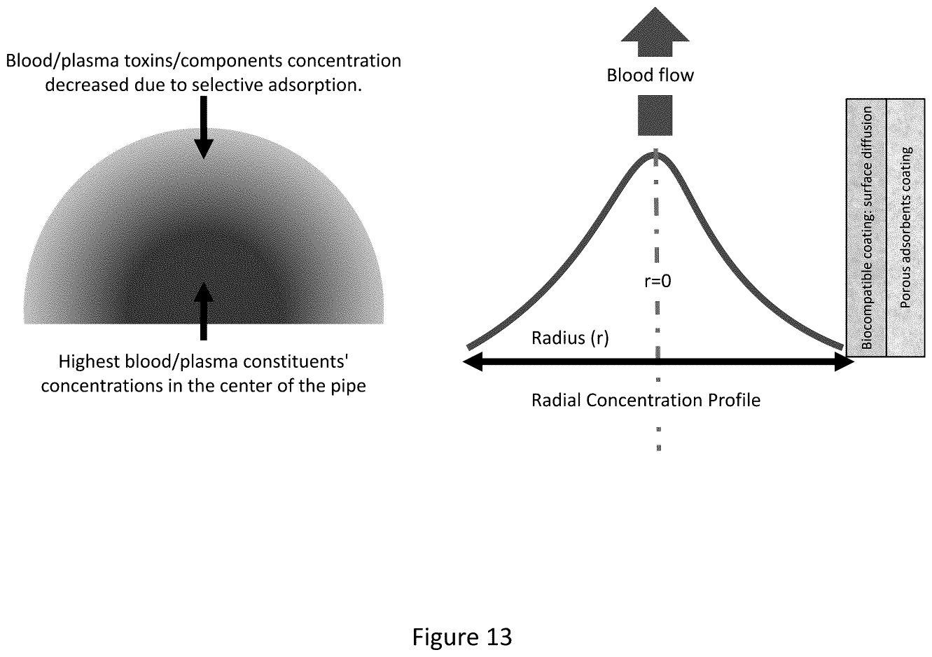

As with PFRs, the inner surface of hemodialysis and hemoperfusion tubing may be functionalized to further improve the procedures. The inner wall of the tubing is coated with an adsorbent layer of MnO₂, MnO₂-adhesive, activated carbon, adsorptive polymer (e.g., sevelamer), or a combination thereof to remove heavy metals, hydrophobic toxins, uremic toxins, phosphate, and other contaminants. An additional biocompatible porous layer is applied over the adsorbent layer to prevent direct contact of blood cells with the adsorbent layer (FIG. 12). In this manner, even before the traditional purification step of hemodialysis or hemoperfusion, heavy metals, toxins, and contaminants are partially removed from the blood by the tubing (FIG. 13).

### Example 7: Iron- and Lanthanide-Containing Surface Coatings

Nanosized iron oxide was prepared by dissolving 200 mg iron chloride in 19.5 mL distilled water. Sodium hydroxide (500 μL, 1 M) was added to the solution, and the solution sealed in a 30 mL reaction vessel. The reaction vessel was sealed with a septa cap and placed in a microwave reactor (Monowave 400, Anton Parr). The solution was then quickly heated to 80° C. in a period of ~5 s and the temperature was held for 30 min. The reaction solution was then quickly cooled down to room temperature. The products were then collected via centrifugation at 6,000 rpm for 10 min followed by washing with DI water.

Iron oxide surface coatings are prepared by culturing an acidophile with the prepared nanosized iron and a surface to be coated, including pipes and plug flow reactors.

The disclosures of each and every patent, patent application, and publication cited herein are hereby incorporated by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of forming an essentially uniform coating of manganese (IV) oxide (MnO₂) on a surface comprising:
a) contacting a surface with a solution comprising one or more manganese (II) salts; and
b) contacting the surface with a solution comprising one or more permanganate salts to create a MnO₂ coating.

2. The method of claim 1, wherein the method further comprises a step c) repeating steps a) and b) until the MnO₂ coating is of a desired thickness.

3. The method of claim 1, wherein the one or more manganese (II) salts is manganese (II) nitrate (Mn(NO₃)₂) or potassium permanganate (KMnO₄).

4. The method of claim 3, wherein the solution comprising Mn(NO₃)₂ or KMnO₄ is at a concentration of about 20 g/L.

5. (canceled)

6. (canceled)

7. The method of claim 1, wherein the solution comprising one or more manganese (II) salts and the solution comprising one or more permanganate salts are at about 50° C.

8. (canceled)

9. The method of claim 1, wherein the solution comprising one or more manganese (II) salts has an Mn²⁺ concentration between about 0.05 M and about 0.5 M; or
the solution comprising one or more permanganate salts has an MnO₄⁻ concentration between about 0.05 M and about 0.5 M.

10. (canceled)

11. The method of claim 1, wherein the surface comprises one or more materials selected from the group consisting of plastic, glass, and metal.

12. The method of claim 11, wherein the plastic is polyvinyl chloride (PVC); or
the metal is copper.

13. (canceled)

14. A method of forming an essentially uniform coating of manganese (IV) oxide (MnO₂) on a surface comprising:
a) contacting a surface with a solution comprising one or more manganese (II) salts, one or more permanganate salts, and one or more stabilizers; and
b) allowing the surface to dry to create a MnO₂ coating.

15. The method of claim 14, wherein the method further comprises a step c) repeating steps a) and b) until the MnO₂ coating is of a desired thickness.

16. The method of claim 14, wherein the one or more Mn (II) salts is Mn(NO₃)₂ or KMnO₄.

17. (canceled)

18. The method of claim 15, wherein the one or more stabilizers are selected from surfactants, polymers, and combinations thereof.

19. (canceled)

20. (canceled)

21. The method of claim 15, wherein the method further comprises applying to the surface one or more carbon-based adsorbents, wherein the carbon-based adsorbents are selected from the group consisting of carbon, charcoal, graphene, graphene oxide, graphitic composites, carbon tubes, and carbon fibers; or
wherein the method further comprises applying to the surface one or more biocompatible polymers or resins, wherein the biocompatible polymer or resin is selected from the group consisting of cyanoacrylates, fibrinogen, thrombin, polyethylene glycol (PEG), silicone, polyurethanes, methyl methacrylate, gelatin-resorcinol-formaldehyde (GRF) glue, albumin, glutaraldehyde, acrylics, and epoxy resins; or
step a) further comprises exposing the solution to microwaves before contacting the surface with the solution.

22. (canceled)

23. (canceled)

24. A method of preventing or minimizing bacterial contamination on a surface comprising covering the surface with a MnO₂ coating produced according to the method of claim 1.

25. A method of reducing heavy metal contamination in an aqueous environment comprising placing an object having a surface comprising a MnO₂ coating produced according to the method of claim 1 in the aqueous environment.

26. (canceled)

27. A plug flow reactor having pipes, tubes, and/or lines having inner surfaces partially or fully coated with a MnO₂ coating produced according to the method of claim 1.

28. (canceled)

29. (canceled)

30. A plug flow reactor wherein the plug flow reactor comprises a static mixer having a surface, wherein the surface of the static mixer is partially or fully coated with a MnO₂ coating produced according to the method of claim 1.

31. A dialysis line having an inner surface, wherein the inner surface of the dialysis line is coated with an adsorbent layer comprising an MnO₂ coating produced according to the method of claim 1.

32. The dialysis line of claim 31, wherein the adsorbent layer further comprises one or more of:
a) one or more selected from the group consisting of activated charcoal, amberlite, XAD resins, zeolites, silica gel, polyacrylate hydrogels, cellulose acetate-coated activated charcoal, chitosan, activated alumina, modified graphite, and graphene; and
b) an adsorptive polymer selected from the group consisting of sevelamer, polystyrene sulfonate, and cholestyramine.

33. (canceled)

34. The dialysis line of claim 32, wherein:
the inner surface of the dialysis line further comprises a biocompatible porous layer comprising one or more selected from the group consisting of: polystyrene, polypropylene, polyvinylchloride, polyethylene, polyurethane, polycarbonate, polyethylene terephthalate, polyether ether ketone, PEG, PPG, polysulfone (PSU), polyethersulfone (PES), polyacrylonitrile (PAN), polyamide, cellulose acetate, modified cellulose, hemophan, polyvinylpyrrolidone (PVP), and poly(methyl methacrylate) (PMMA), polyethylene glycol diacrylate, poly(lactic-co-glycolic acid) (PLGA), polydimethylsiloxane (PDMS), polyvinyl alcohol (PVA), polyacrylamide, polytetrafluoroethylene, and combinations thereof,
the inner surface of the dialysis line further comprises a biocompatible porous layer having an average pore size between about 10 nm and about 6 μm; or
the adsorbent layer is impregnated with one or more active agents.

35.-39. (canceled)
